(19) United States
(12) Patent Application Publication
(10) Pub. No.: US 2012/0096600 A1
(43) Pub. Date: Apr. 19, 2012

(54) METHODS AND MEANS FOR OBTAINING PLANTS WITH ENHANCED GLYPHOSATE TOLERANCE

(76) Inventors: Rene Ruiter, Heusden (BE); Frank Meulewaeter, Merelbeke (BE); Chantal Vanderstraeten, Gent (BE)

(21) Appl. No.: 13/378,779

(22) PCT Filed: Jun. 24, 2010
(86) PCT No.: PCT/EP2010/003797

§ 371 Date: Dec. 16, 2011

Related U.S. Application Data
(60) Provisional application No. 61/270,150, filed on Jul. 6, 2009.

(30) Foreign Application Priority Data
Jul. 1, 2009 (EP) 09075283.3

(51) Int. Cl. A01H 5/00; C07H 21/04; A01H 5/10; C12N 5/10

(52) U.S. Cl. 800/300; 435/419; 536/23.2

(57) ABSTRACT

The present invention relates to plants with a chimeric DNA molecule encoding a glyphosate tolerant EPSPS enzyme under the control of a plant constitutive promoter and a replacement histone intron 1, thereby conferring enhanced glyphosate tolerance to said plants.

### FIELD OF THE INVENTION

The invention relates to the field of herbicide tolerant plants, more specifically plants, such as Brassica oilseed plants, comprising a chimeric DNA molecule which directs quantitative and qualitative expression of a glyphosate tolerant 5-enolpyruvylshikimate-3-phosphate synthase (EPSPS), said chimeric DNA molecule thereby conferring enhanced tolerance on said plants to herbicides inhibiting said EPSPS.

### BACKGROUND OF THE INVENTION

N-phosphonomethylglycine, also known as glyphosate, is a well-known herbicide that has activity on a broad spectrum of plant species. Glyphosate is phytotoxic due to its inhibition of the shikimic acid pathway, which provides a precursor for the synthesis of aromatic amino acids. Glyphosate inhibits the class I 5-enolpyruvylshikimate-3-phosphate synthase (EPSPS) found in plants and some bacteria. Glyphosate tolerance in plants can be achieved by the expression of a modified class I EPSPS that has lower affinity for glyphosate, yet still retains its catalytic activity in the presence of glyphosate. Genes encoding glyphosate-tolerant EPSPS enzymes are well known in the art e.g. in patent application EP 0 837 944 and U.S. Pat. No. 6,566,587. Glyphosate tolerance in plants may also be achieved by expression of EPSPS enzymes which exhibit tolerance to glyphosate including class II or class III EPSPS enzymes.

The extent of glyphosate tolerance in plants is essentially based on the quality and the quantity of expression of the EPSPS enzyme i.e. the expression of EPSPS in sufficient quantities in the appropriate tissues at the appropriate developmental stage. These parameters of quality and quantity of expression are controlled in part by the regulatory elements introduced into the expression cassette directing EPSPS expression. The regulatory elements essential to an expression cassette include the promoter regulatory sequence and the terminator regulatory sequence. To further enhance expression, expression cassettes can also contain either one or more or all of the following elements selected from a leader sequence or 5′UTR, a signal peptide or a transit peptide, or a transcription activator element or enhancer. Various methods have been described in the art to improve expression of a glyphosate tolerance chimeric gene in plants, particularly crop plants such as oilseed rape.

WO97/004114 describes a chimeric gene for transforming plants. The gene includes in the transcription direction at least one promoter region, one transgene and one regulatory region consisting of at least one intron 1 of the non-coding 5′ region of a plant histone gene enabling expression of the proteins in rapid growth regions.

WO01/44457 discloses multiple plant expression constructs containing various actin intron sequences in combination with the PeFMV promoter for enhanced transgene expressing, including EPSPS.

In WO 07/098,042 combinations of monocot promoters with dicot introns from EF1, Act and ASP genes directing expression of a.o. EPSPS, glyphosate oxidoreductase (GOX) and glyphosate acetyl transferase are described.

Enhanced expression of CP4 EPSPS by the CaMV 35S promoter in combination with an EF1α intron in cotton is reported by Chen et al. (2006, Plant Biotechnol J. 4(5):477-87).

Nevertheless, further improvement of glyphosate tolerance in crop plants, particularly oilseed rape plants is desirable, and alternative chimeric genes or combinations thereof which confer increased tolerance are still a need.

This invention makes a significant contribution to the art by providing plants comprising a combination of a constitutive promoter with a replacement histone intron directing the expression of a glyphosate tolerant EPSPS enzyme from a EPSPS coding region, such as a EPSPS coding region wherein the codon usage has been optimized to reflect codon usage in oilseed rape. Inclusion of a histone intron in the glyphosate tolerance chimeric genes, particularly in combination with a codon usage optimized EPSPS coding region as herein described, provides an alternative approach to obtain efficient glyphosate tolerance in crop plants, particularly oilseed rape plants.

This problem is solved as herein after described in the different embodiments, examples and claims.

### SUMMARY OF THE INVENTION

Generally, the present invention relates to plants with enhanced glyphosate tolerance by increasing the quality and the quantity of expression of a glyphosate tolerant EPSPS enzyme which is directed by a plant expressible constitutive promoter and an intron 1 of a replacement histone gene. The invention also provides chimeric DNA molecules or genes, as well as methods of treating the plants of the invention to generate glyphosate tolerant plants.

In a first embodiment, plants are provided comprising a chimeric DNA molecule, wherein the chimeric DNA molecule comprises the following operably linked DNA fragments:
a) a plant-expressible constitutive promoter;
b) a DNA region encoding a 5′UTR;
c) a DNA region encoding an intron 1 of a plant replacement histone gene;
d) a DNA region encoding a transit peptide;
e) a DNA region encoding a glyphosate-tolerant 5-enolpyruvylshikimate-3-phosphate synthase (EPSPS); and
f) a 3′ transcription termination and polyadenylation region.

According to another embodiment of the invention, the plant expressible constitutive promoter comprises the cauliflower mosaic virus (CaMV) 35S promoter.

In yet another embodiment, the plants according to the invention additionally comprise a second chimeric DNA molecule, said second chimeric DNA molecule comprising the following operably linked DNA fragments:
a) a promoter sequence of the histone H4 gene of Arabidopsis thaliana;
b) a DNA region encoding an intron 1 of a plant replacement histone gene;
c) a DNA region encoding a transit peptide;
d) a DNA region encoding a glyphosate-tolerant EPSPS; and
e) a 3′ transcription termination and polyadenylation region.

In a further embodiment, the histone H4 promoter sequence comprises the full length H4A748 promoter, more specifically the nucleotide (nt) sequence from position 6166 to 7087 of SEQ ID no. 6.

According to another embodiment, the intron 1 encoding DNA region comprises a nucleotide sequence selected from the group consisting of genbank accession number X60429.1 or U09458.1.

In a further embodiment of the invention, the nucleotide sequence of the DNA region encoding the glyphosate tolerant EPSPS is adapted to Brassica napus codon usage.

In yet another embodiment the plants of the invention are Brassica plants, more specifically oilseed rape, even more specifically Brassica napus, Brassica rapa, Brassica campestris or Brassica juncea.

The invention also provides plant cells and seeds of the plants of the invention comprising the chimeric genes, as well as the chimeric DNA molecules themselves and cloning and/or expression vectors comprising those genes.

The invention also relates to a method for treating plants with an EPSPS inhibiting herbicide, more specifically glyphosate, wherein said plant is tolerant to an application of at least 2.0 kg active ingredient/ha, although clearly lower concentrations of a.i. may be applied.

### BRIEF DESCRIPTION OF THE DRAWINGS

FIG. 1: Panel A: Schematic representation of the different glyphosate tolerance chimeric genes and combinations thereof. P35S-2: CaMV 35S promoter; cab22L: leader sequence of the chlorophyl a/b binding protein gene from Petunia hybrida; TpotPc-1Pc: optimized transit peptide, containing sequence of the RuBisCO small subunit genes of Zea mays and Helianthus annuus, adapted to Brassica napus codon usage; 2mEPSPS-1 Pa: double-mutant 5-enol-pyruvylshikimate-3-phosphate synthase gene of Zea mays, adapted to Brassica napus codon usage; 3′ nos: 3′UTR of the nopaline synthase gene from the T-DNA of pTiT37; Ph4a748-NarI: Nan fragment of the promoter of the histone H4 gene of Arabidopsis thaliana; intron1h3: first intron of gene II of the histone H3.III variant of Arabidopsis; 3′ his: 3′UTR of the histone H4 gene of Arabidopsis thaliana; Ph4a748: full length promoter of the histone H4 gene of Arabidopsis thaliana.

Panel B: Transgenic Brassica napus plants containing glyphosate tolerance chimeric genes herein described 10 days after spraying with 2.0 kg/ha a.i. glyphosate.

### DETAILED DESCRIPTION OF THE INVENTION

The present invention is based on the observation that inclusion of an intron 1 of a replacement histone gene from a plant in a chimeric gene comprising a constitutive promoter, such as CaMV35S promoter, significantly improved the glyphosate tolerance of transgenic plants comprising such chimeric genes when compared to transgenic plants comprising a corresponding chimeric gene lacking such intron sequence. Furthermore, the inventors have observed that use of an EPSPS coding region optimized for codon usage in oilseed rape plants provided better glyphosate tolerance, than for plants wherein a similar EPSPS coding region derived from a monocotyledonous plant was used. The glyphosate tolerance can be further improved by including a second glyphosate tolerance chimeric gene wherein a promoter such as a histone H4 promoter (H4A748) is operably linked to an intron 1 of a replacement histone gene and an EPSPS coding region. In contrast to scientific reports of previous observations (Chaubet-Gigot et al., 2001 Plant Mol. Biol. 45(1):17-30) wherein a combination of a truncated Nan fragment of the H4A748 promoter and a replacement histone intron 1 was described as superior over a combination of the full length H4A748 promoter promoter and a replacement histone intron 1 (as described in WO1997/004114), it was surprisingly found that in combination with EPSPS the full length version of the promoter conferred better glyphosate tolerance to plants containing such chimeric molecules than the truncated version.

Accordingly, in one embodiment, the invention provides a glyphosate tolerant plant containing a chimeric DNA molecule, wherein the chimeric DNA molecule comprises the following operably linked DNA fragments:
a) a plant-expressible constitutive promoter;
b) a DNA region encoding a 5′UTR;
c) a DNA region encoding an intron 1 of a plant replacement histone gene;
d) a DNA region encoding a transit peptide;
e) a DNA region encoding a glyphosate-tolerant 5-enolpyruvylshikimate-3-phosphate synthase (EPSPS); and
f) a 3′ transcription termination and polyadenylation region.

As used herein “a chimeric DNA molecule” is intended to mean a DNA molecule consisting of multiple linked DNA fragments of various origins. By way of example, a chimeric DNA molecule can comprise a viral promoter linked to a plant coding sequence. The term chimeric gene or chimeric DNA molecule is also interchangeably used with the term transgene or recombinant DNA molecule. As used herein, the term chimeric gene, molecule refers to a DNA molecule wherein the different elements originally are not found in this arrangement in nature and are or have been man-made.

As used herein “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a DNA region which is functionally or structurally defined may comprise additional DNA regions etc.

The expression “operably linked” means that said elements of the chimeric gene are linked to one another in such a way that their function is coordinated and allows expression of the coding sequence. By way of example, a promoter is functionally linked to a coding sequence when it is capable of ensuring transcription and ultimately expression of said coding sequence.

As used herein, a “plant expressible constitutive promoter” is a promoter capable of functioning in plant cells and plants directing high levels of expression in most cell types (in a spatio-temporal independent manner). Examples include bacterial promoters, such as that of octopine synthase (OCS) and nopaline synthase (NOS) promoters from Agrobacterium, but also viral promoters, such as that of the cauliflower mosaic virus (CaMV) 35S or 19S RNAs genes (Odell et al., 1985, Nature. 6; 313(6005):810-2), promoters of the cassava vein mosaic virus (CsVMV; WO 97/48819), the sugarcane bacilliform badnavirus (ScBV) promoter (Samac et al., 2004, Transgenic Res. 13(4):349-61), the figwort mosaic virus (FMV) promoter (Sanger et al., 1990, Plant Mol. Biol. 14(3):433-43) and the subterranean clover virus promoter No 4 or No 7 (WO 96/06932). Among the promoters of plant origin, mention will be made of the promoters of the Rubisco small subunit promoter (U.S. Pat. No. 4,962,028), the ubiquitin promoters of Maize, Rice and sugarcane, the Rice actin 1 promoter (Act-1) and the Maize alcohol dehydrogenase 1 promoter (Adh-1) (from http://www.patentlens.net/daisy/promoters/242.html).

According to another embodiment of the invention, the plant expressible constitutive promoter comprises the cauliflower mosaic virus (CaMV) 35S promoter, more specifically the nucleotide sequence of SEQ ID 2 from nucleotide (nt) position 2352 to 2770.

Introns are intervening sequences present in the pre-mRNA but absent in the mature RNA following excision by a precise splicing mechanism. The ability of natural introns to enhance gene expression, a process referred to as intron-mediated enhancement (IME), has been known in various organisms, including mammals, insects, nematodes and plants (WO 07/098,042, p 11-12). IME is generally described as a posttranscriptional mechanism leading to increased gene expression by stabilization of the transcript. The intron is required to be positioned between the promoter and the coding sequence in the normal orientation. However, some introns have also been described to affect translation, to function as promoters or as position and orientation independent transcriptional enhancers (Chaubet-Gigot et al., 2001, Plant Mol. Biol. 45(1):17-30, p 27-28).

Examples of genes containing such introns include the maize sucrose synthase gene (Clancy and Hannah, 2002, Plant Physiol. 130(2):918-29), the maize alcohol dehydrogenase-1 (Adh-1) and Bronze-1 genes (Callis et al. 1987 Genes Dev. 1(10):1183-200; Mascarenhas et al. 1990, Plant Mol. Biol. 15(6):913-20), the replacement histone H3 gene from alfalfa (Keleman et al. 2002 Transgenic Res. 11(1):69-72) and either replacement histone H3 (histone H3.3-like) gene of Arabidopsis thaliana (Chaubet-Gigot et al., 2001, Plant Mol. Biol. 45(1):17-30).

As used herein, an “intron 1 of a plant replacement histone gene” relates to the intron in the 5′ untranslated region (UTR) of replacement histone encoding genes. Replacement histones function to repair nucleosomal chromatin structure across transcribed genes (Waterborg et al., 1993, J Biol. Chem. 5; 268(7):4912-7), in contrast to replication histones, which mediate the assembly of nucleosomes in S-phase cells and transcriptional activation of such histone genes is restricted to the S-phase (Atanassova et al., 1992, Plant J. 1992 2(3):291-300).

According to another embodiment of the invention, the nucleotide sequence encoding an intron 1 of a histone replacement gene is derived form the histone H3.III variant genes of Arabidopsis thaliana or from the histone H3.2 gene of Medicago sativa. More specifically, the intron 1 encoding DNA region may comprise a nucleotide sequence selected from the group consisting of genbank accession number X60429.1 or U09458.1 (herein incorporated by reference). More specifically, the intron 1 encoding DNA region comprises nt 692 to 1100 or nt 2984 to 3064 of SEQ ID no. 9 or nt 555 to 668 of SEQ ID no. 10.

According to the invention, the term “EPSPS” is intended to mean any native or mutated 5-enolpyruvylshikimate-3-phosphate synthase enzyme, the enzymatic activity of which consists in synthesizing 5-O— (1-carboxyvinyl)-3-phosphoshikimate from phosphoenolpyruvate (PEP) and 3-phosphoshikimate (EC 2.5.1.19; Morell et al., 1967, J. Biol. Chem. 242:82-90). In particular, said EPSPS enzyme may originate from any type of organism. An EPSPS enzyme suitable for the invention also has the property of being tolerant with respect to herbicides of the phosphonomethylglycine family, in particular with respect to glyphosate.

Sequences encoding EPSPSs which are naturally tolerant, or are used as such, with respect to herbicides of the phosphonomethylglycine family, in particular glyphosate, are known. By way of example, mention may be made of the sequence of the AroA gene of the bacterium Salmonella typhimurium (Comai et al., 1983, Science 221:370-371), the sequence of the CP4 gene of the bacterium Agrobacterium sp. (WO 92/04449), or the sequences of the genes encoding Petunia EPSPS (Shah et al., 1986, Science 233:478-481), tomato EPSPS (Gasser et al., 1988, J. Biol. Chem. 263:4280-4289), or eleusine EPSPS (WO 01/66704).

Sequences encoding EPSPSs made tolerant to glyphosate by mutation are also known. By way of example, mention may be made of the sequences of the genes encoding a mutated AroA EPSPS (Stalker et al., 1985, J. Biol. Chem. 260(8):4724-4728), or a mutated E. coli EPSPS (Kahrizi et al., 2007, Plant Cell Rep. 26(1):95-104). Examples of mutated EPSPS enzymes of plant origin include a double mutant (2m) EPSPS with an alanine to glycine substitution between positions 80 and 120 and a threonine to alanine substitution between positions 170 and 210 (e.g. EP 0293358, WO 92/06201) and various double mutants with aminoacid substitutions at position 102 and 106 (e.g. U.S. Pat. No. 6,566,587, WO04/074443).

Sequences encoding EPSPSs tolerant to glyphosate further include those described in WO2008/100353, WO2008/002964, WO2008/002962, WO2007/146980, WO2007/146765, WO2007/082269, WO2007/064828 or WO2006/110586.

According to another embodiment of the invention, a sequence of a gene encoding a glyphosate-tolerant EPSPS may be a sequence encoding the maize EPSPS described in patent application EP 0837944, comprising a first mutation replacing the threonine amino acid at position 102 with isoleucine, and a second mutation replacing the proline amino acid at position 106 with serine. More specifically, said EPSPS encoding DNA region encodes the amino acid sequence of SEQ ID no. 8. Due to the strong sequence homology between EPSPSs, and more particularly between plant EPSPSs, a rice EPSPS carrying the same mutations has also been described in patent applications WO 00/66746 and WO 00/66747. In general, any EPSPS, and the genes encoding them, carrying the threonine/isoleucine and proline/serine mutations described above, whatever the relative position of these amino acids with respect to positions 102 and 106 of maize EPSPS, can be used in the present invention. To apply this principle, those skilled in the art will be readily able to find the two amino acids to be mutated in any EPSPS sequence by using standard techniques of sequence alignment.

It is well known that different organisms often show particular preferences for one of the several codons that encode the same amino acid. It is thought that the presence of optimal codons may help to achieve faster translation rates and high accuracy. Lutz et al (2001, Plant Physiol. 125(4):1585-90) report enhanced expression of a codon-optimized bacterial bar gene in tobacco. Peng et al. (2006, Plant Cell Rep. 25(2):124-32) demonstrate that the expression of an Aspergillus niger derived transgene in canola can be improved by adapting the sequence according to Brassica codon usage. Nevertheless, it remains unpredictable whether such strategy will work in a particular situation. For example, WO 08/024,372 reports that codon-optimization of the pullulanase coding region from Bacillus deramificans does not result in increased pullulanase production in Bacillus licheniformis. Further, Gregersen et al. (2005, Transgenic Res. 14(6):887-905) describe that the codon-optimization of an A. fumigatus phytase gene for expression in wheat had no significant effects on the overall gene expression.

However, as herein described, further improvement of expression of glyphosate tolerance chimeric genes in plants, such as oilseed rape plants, can be achieved by optimizing the sequence encoding the protein to be expressed according to the codon usage of the plant intended for overexpression.

Thus, in another embodiment, the glyphosate-tolerant EPSPS encoding nucleotide sequence has been optimized for Brassica napus codon usage in order to fulfill the following criteria:
a) the overall percentages of codon usage for each aminoacid correspond to those as observed for Brassica napus;
b) the nucleotide sequence has an AT content greater than 54%;
c) the nucleotide sequence does not comprise 5′ or 3′ cryptic splice sites or a nucleotide sequence selected from the group consisting of AAGGTAAGT, AAGGTAA, AGGTAA or TGCAG; and
d) the nucleotide sequence does not comprise polyadenylation signals or a nucleotide sequence selected from the group consisting of CATAAA, AACCAA, ATTAAT, AAAATA, AATTAA, AATACA.

It will be clear to the person skilled in the art that for cloning purposes, the nucleotide sequence may be modified with regard to presence or absence of recognition sequences for certain restriction enzymes, while still fulfilling the above mentioned criteria.

According to a specific embodiment, the glyphosate-tolerant EPSPS encoding nucleotide sequence comprises nt 997-2334 of SEQ ID no. 1.

It will also be clear to the person skilled in the art that the exemplified nucleotide sequence may be further modified, while still encoding a glyphosate tolerant EPSPS enzyme by 100 nt, 75 nt, 50 nt, 40 nt, 30 nt, 20 nt, 10 nt or 5 nt, while still fulfilling the above mentioned criteria.

Thus, in another embodiment, the glyphosate-tolerant EPSPS encoding nucleotide sequence has been optimized for Brassica napus codon usage.

More specifically, said EPSPS encoding DNA region comprises nt 997-2334 of SEQ ID no. 1.

In another embodiment, the plant of the invention further comprises in its chimeric DNA molecule operably linked a DNA region encoding a 5′ untranslated region (UTR).

As used herein, a 5′UTR, also referred to as leader sequence, is a particular region of a messenger RNA (mRNA) located between the transcription start site and the start codon of the coding region. It is involved in mRNA stability and translation efficiency. For example, the 5′ untranslated leader of a petunia chlorophyll a/b binding protein gene downstream of the 35S transcription start site can be utilized to augment steady-state levels of reporter gene expression (Harpster et al., 1988, Mol Gen Genet. 212(1):182-90). WO95/006742 describes the use of 5′ non-translated leader sequences derived from genes coding for heat shock proteins to increase transgene expression.

In a further embodiment of the invention, the DNA region encoding a 5′UTR may comprise the leader sequence of the chlorophyl a/b binding protein gene from Petunia hybrida, more specifically nt 2283-2351 of SEQ ID no. 2.

According to the invention, the chimeric DNA molecule also comprises a subcellular addressing sequence encoding a transit peptide or signal peptide. Such a sequence, located upstream or downstream of the nucleic acid sequence encoding the EPSPS, makes it possible to direct said EPSPS specifically into a cellular compartment of the host organism.

According to a specific embodiment, the transit peptide comprises, in the direction of transcription, at least one signal peptide sequence of a plant gene encoding a signal peptide directing transport of a polypeptide to a plastid, a portion of the sequence of the mature N-terminal part of a plant gene produced when the first signal peptide is cleaved by proteolytic enzymes, and then a second signal peptide of a plant gene encoding a signal peptide directing transport of the polypeptide to a sub-compartment of the plastid. The signal peptide sequence is preferably derived from a gene for the small subunit of ribulose-1,5-bisphosphate carboxylase/oxygenase (RuBisCO) according to EP0508909. More specifically, the transit peptide encoding DNA region encodes the aminoacid sequence of SEQ ID no. 7.

According to yet another embodiment, the nucleotide sequence encoding the transit peptide has also been optimized for Brassica napus codon usage, more specifically comprising nt 2335-2706 of SEQ ID no. 1.

It is believe that the specific transcription termination and polyadenylation region which can be used according to the invention is immaterial and any such sequence known in the art may be used with similar effect. As non-limiting examples, the nos terminator sequence of the gene encoding Agrobacterium tumefaciens nopaline synthase (Bevan et al., 1983, Nucleic Acids Res. 11(2); 369-385), or the his terminator sequence of a histone gene as described in application EP 0 633 317 are mentioned.

The present invention also relates to plants additionally containing a second chimeric DNA molecule, wherein the second chimeric DNA molecule comprises the following operably linked DNA fragments;
a) a promoter sequence of the histone H4 gene of Arabidopsis thaliana;
b) a DNA region encoding an intron 1 of a plant replacement histone gene;
c) a DNA region encoding a transit peptide;
d) a DNA region encoding a glyphosate-tolerant EPSPS; and
e) a 3′ transcription termination and polyadenylation region.

The promoter of the histone H4 gene of Arabidopsis thaliana (H4A748) drives strong preferential expression in an S-phase and meristem specific pattern, while remaining basal expression in non-dividing cells (Atanassova et al., 1992, Plant J. 1992 2(3):291-300). However, addition of the 5′UTR intron of either replacement histon H3 gene of Arabidopsis thaliana to this cell cycle-dependent promoter results in high, meristem independent reporter gene expression. Particularly, a truncated NarI fragment of this promoter in combination with the intron 1 induces an even 3-4 fold higher reporter gene expression level in buds and roots than the full length H4A748 promoter with the intron (Chaubet-Gigot et al., 2001 Plant Mol. Biol. 45(1):17-30, FIG. 4).

According to another embodiment, the promoter sequence of the histone H4 gene of Arabidopsis thaliana comprises the full length H4A748 sequence, more specifically nt 6166-7087 of SEQ ID no. 6.

In further embodiments, the second chimeric DNA molecule also comprises a DNA region encoding an intron 1 of a plant replacement histone gene, a DNA region encoding a transit peptide, a DNA region encoding a glyphosate-tolerant EPSPS and a 3′ transcription termination and polyadenylation region. These DNA regions are similar as described elsewhere in this application.

According to another embodiment, the plant of the invention is a Brassica plant, more preferably an oilseed rape plant. As used herein “oilseed rape” refers to any one of the species Brassica napus, Brassica rapa, Brassica campestris or Brassica juncea.

However, it will be clear to the skilled artisan that the methods and means described herein are believed to be suitable for all plant cells and plants, both dicotyledonous and monocotyledonous plant cells and plants including but not limited to cotton, Brassica vegetables, oilseed rape, wheat, corn or maize, barley, alfalfa, peanuts, sunflowers, rice, oats, sugarcane, soybean, turf grasses, barley, rye, sorghum, sugar cane, vegetables (including chicory, lettuce, tomato, zucchini, bell pepper, eggplant, cucumber, melon, onion, leek), tobacco, potato, sugarbeet, papaya, pineapple, mango, Arabidopsis thaliana, but also plants used in horticulture, floriculture or forestry (poplar, fir, eucalyptus etc.).

t is also an embodiment of the invention to provide plant cells containing the chimeric DNA molecules according to the invention. Gametes, seeds, embryos, either zygotic or somatic, progeny or hybrids of plants comprising the chimeric DNA molecules of the present invention, which are produced by traditional breeding methods, are also included within the scope of the present invention.

Another object of the invention are the chimeric DNA molecules as herein described or a cloning and/or expression vector for transforming plants, comprising such chimeric DNA molecule.

The chimeric DNA molecules according to the invention can be stably inserted in a conventional manner into the nuclear genome of a single plant cell, and the so transformed plant cell can be used in a conventional manner to produce a transformed plant with enhanced glyphosate tolerance. In this regard, a T-DNA vector, containing the chimeric DNA molecule(s), in Agrobacterium tumefaciens can be used to transform the plant cell, and thereafter, a transformed plant can be regenerated from the transformed plant cell using the procedures described, for example, in EP 0 116 718, EP 0 270 822, WO 84/02913 and published European Patent application EP 0 242 246 and in Gould et al. (1991, Plant Physiol. 95(2):426-434). The construction of a T-DNA vector for Agrobacterium mediated plant transformation is well known in the art. The T-DNA vector may be either a binary vector as described in EP 0 120 561 and EP 0 120 515 or a co-integrate vector which can integrate into the Agrobacterium Ti-plasmid by homologous recombination, as described in EP 0 116 718. Preferred T-DNA vectors each contain a promoter operably linked to the transcribed DNA region between T-DNA border sequences, or at least located to the left of the right border sequence. Border sequences are described in Gielen et al. (1984, EMBO J. 3(4):835-46). Introduction of the T-DNA vector into Agrobacterium can be carried out using known methods, such as electroporation or triparental mating. Of course, other types of vectors can be used to transform the plant cell, using procedures such as direct gene transfer (as described, for example in EP 0223247), pollen mediated transformation (as described, for example in EP 0270356 and WO 85/01856), protoplast transformation as, for example, described in U.S. Pat. No. 4,684,611, plant RNA virus-mediated transformation (as described, for example in EP 0067553 and U.S. Pat. No. 4,407,956), liposome-mediated transformation (as described, for example in U.S. Pat. No. 4,536,475), and other methods such as the recently described methods for transforming certain lines of corn (e.g., U.S. Pat. No. 6,140,553; Fromm et al., 1990, Biotechnology (N Y). 8(9):833-9; Gordon-Kamm et al., 1990, Plant Cell. 1990 2(7):603-618) and rice (Shimamoto et al., 1989, Tanpakushitsu Kakusan Koso. 34(14):1873-8) and the method for transforming monocots generally (WO 92/09696). For cotton transformation, especially preferred is the method described in PCT patent publication WO 00/71733. For rice transformation, reference is made to the methods described in WO 92/09696, WO 94/00977 and WO 95/06722. The resulting transformed plant can be used in a conventional plant breeding scheme to produce more transformed plants with increased glyphosate tolerance.

In another embodiment, a method for treating the plants of the invention with an EPSPS-inhibiting herbicide, more specifically glyphosate, is provided. Even more specifically, the plants of this method are tolerant to applications of 2.0 kg/ha glyphosate.

In another embodiment, the use of chimeric DNA molecules of the invention to obtain glyphosate tolerant plants is provided.

Plants according to the invention may be treated with at least one of the following chemical compounds The plants and seeds according to the invention may be further treated with a chemical compound, such as a chemical compound selected from the following lists:
a. Fruits/Vegetables Herbicides: Atrazine, Bromacil, Diuron, Glyphosate, Linuron, Metribuzin, Simazine, Trifluralin, Fluazifop, Glufosinate, Halosulfuron Gowan, Paraquat, Propyzamide, Sethoxydim, Butafenacil, Halosulfuron, Indaziflam
b. Fruits/Vegetables Insecticides: Aldicarb, Bacillus thuriengiensis, Carbaryl, Carbofuran, Chlorpyrifos, Cypermethrin, Deltamethrin, Abamectin, Cyfluthrin/beta-cyfluthrin, Esfenvalerate, Lambda-cyhalothrin, Acequinocyl, Bifenazate, Methoxyfenozide, Novaluron, Chromafenozide, Thiacloprid, Dinotefuran, Fluacrypyrim, Spirodiclofen, Gamma-cyhalothrin, Spiromesifen, Spinosad, Rynaxypyr, Cyazypyr, Triflumuron, Spirotetramat, Imidacloprid, Flubendiamide, Thiodicarb, Metaflumizone, Sulfoxaflor, Cyflumetofen, Cyanopyrafen, Clothianidin, Thiamethoxam, Spinotoram, Thiodicarb, Flonicamid, Methiocarb, Emamectin-benzoate, Indoxacarb, Fenamiphos, Pyriproxifen, Fenbutatin-oxid
c. Fruits/Vegetables Fungicides: Ametoctradin, Azoxystrobin, Benthiavalicarb, Boscalid, Captan, Carbendazim, Chlorothalonil, Copper, Cyazofamid, Cyflufenamid, Cymoxanil, Cyproconazole, Cyprodinil, Difenoconazole, Dimetomorph, Dithianon, Fenamidone, Fenhexamid, Fluazinam, Fludioxonil, Fluopicolide, Fluopyram, Fluoxastrobin, Fluxapyroxad, Folpet, Fosetyl, Iprodione, Iprovalicarb, Isopyrazam, Kresoxim-methyl, Mancozeb, Mandipropamid, Metalaxyl/mefenoxam, Metiram, Metrafenone, Myclobutanil, Penconazole, Penthiopyrad, Picoxystrobin, Propamocarb, Propiconazole, Propineb, Proquinazid, Prothioconazole, Pyraclostrobin, Pyrimethanil, Quinoxyfen, Spiroxamine, Sulphur, Tebuconazole, Thiophanate-methyl, Trifloxystrobin
d. Cereals herbicides: 2.4-d, amidosulfuron, bromoxynil, carfentrazone-e, chlorotoluron, chlorsulfuron, clodinafop-p, clopyralid, dicamba, diclofop-m, diflufenican, fenoxaprop, florasulam, flucarbazone-na, flufenacet, flupyrsulfuron-m, fluoroxypyr, flurtamone, glyphosate, iodosulfuron, ioxynil, isoproturon, mcpa, mesosulfuron, metsulfuron, pendimethalin, pinoxaden, propoxycarbazone, prosulfocarb, pyroxsulam, sulfosulfuron, thifensulfuron, tralkoxydim, triasulfuron, tribenuron, trifluralin, tritosulfuron
e. Cereals Fungicides: Azoxystrobin, Bixafen, Boscalid, Carbendazim, Chlorothalonil, Cyflufenamid, Cyproconazole, Cyprodinil, Dimoxystrobin, Epoxiconazole, Fenpropidin, Fenpropimorph, Fluopyram, Fluoxastrobin, Fluquinconazole, Fluxapyroxad, Isopyrazam, Kresoxim-methyl, Metconazole, Metrafenone, Penthiopyrad, Picoxystrobin, Prochloraz, Propiconazole, Proquinazid, Prothioconazole, Pyraclostrobin, Quinoxyfen, Spiroxamine, Tebuconazole, Thiophanate-methyl, Trifloxystrobin
f. Cereals Insecticides: Dimethoate, Lambda-cyhalthrin, Deltamethrin, alpha-Cypermethrin, β-cyfluthrin, Bifenthrin, Imidacloprid, Clothianidin, Thiamethoxam, Thiacloprid, Acetamiprid, Dinetofuran, Clorphyriphos, Pirimicarb, Methiocarb, Sulfoxaflor
g. Maize Herbicides: Atrazine, Alachlor, Bromoxynil, Acetochlor, Dicamba, Clopyralid, (S-)Dimethenamid, Glufosinate, Glyphosate, Isoxaflutole, (S-)Metolachlor, Mesotrione, Nicosulfuron, Primisulfuron, Rimsulfuron, Sulcotrione, Foramsulfuron, Topramezone, Tembotrione, Saflufenacil, Thiencarbazone, Flufenacet, Pyroxasulfon
h. Maize Insecticides: Carbofuran, Chlorpyrifos, Bifenthrin, Fipronil, Imidacloprid, Lambda-Cyhalothrin, Tefluthrin, Terbufos, Thiamethoxam, Clothianidin, Spiromesifen, Flubendiamide, Triflumuron, Rynaxypyr, Deltamethrin, Thiodicarb, β-Cyfluthrin, Cypermethrin, Bifenthrin, Lufenuron, Tebupirimphos, Ethiprole, Cyazypyr, Thiacloprid, Acetamiprid, Dinetofuran, Avermectin
i. Maize Fungicides: Azoxystrobin, Bixafen, Boscalid, Cyproconazole, Dimoxystrobin, Epoxiconazole, Fenitropan, Fluopyram, Fluoxastrobin, Fluxapyroxad, Isopyrazam, Metconazole, Penthiopyrad, Picoxystrobin, Propiconazole, Prothioconazole, Pyraclostrobin, Tebuconazole, Trifloxystrobin
j. Rice Herbicides: Butachlor, Propanil, Azimsulfuron, Bensulfuron, Cyhalofop, Daimuron, Fentrazamide, Imazosulfuron, Mefenacet, Oxaziclomefone, Pyrazosulfuron, Pyributicarb, Quinclorac, Thiobencarb, Indanofan, Flufenacet, Fentrazamide, Halosulfuron, Oxaziclomefone, Benzobicyclon, Pyriftalid, Penoxsulam, Bispyribac, Oxadiargyl, Ethoxysulfuron, Pretilachlor, Mesotrione, Tefuryltrione, Oxadiazone, Fenoxaprop, Pyrimisulfan
k. Rice Insecticides: Diazinon, Fenobucarb, Benfuracarb, Buprofezin, Dinotefuran, Fipronil, Imidacloprid, Isoprocarb, Thiacloprid, Chromafenozide, Clothianidin, Ethiprole, Flubendiamide, Rynaxypyr, Deltamethrin, Acetamiprid, Thiamethoxam, Cyazypyr, Spinosad, Spinotoram, Emamectin-Benzoate, Cypermethrin, Chlorpyriphos, Etofenprox, Carbofuran, Benfuracarb, Sulfoxaflor
l. Rice Fungicides: Azoxystrobin, Carbendazim, Carpropamid, Diclocymet, Difenoconazole, Edifenphos, Ferimzone, Gentamycin, Hexaconazole, Hymexazol, Iprobenfos (IBP), Isoprothiolane, Isotianil, Kasugamycin, Mancozeb, Metominostrobin, Orysastrobin, Pencycuron, Probenazole, Propiconazole, Propineb, Pyroquilon, Tebuconazole, Thiophanate-methyl, Tiadinil, Tricyclazole, Trifloxystrobin, Validamycin
m. Cotton Herbicides: Diuron, Fluometuron, MSMA, Oxyfluorfen, Prometryn, Trifluralin, Carfentrazone, Clethodim, Fluazifop-butyl, Glyphosate, Norflurazon, Pendimethalin, Pyrithiobac-sodium, Trifloxysulfuron, Tepraloxydim, Glufosinate, Flumioxazin, Thidiazuron
n. Cotton Insecticides: Acephate, Aldicarb, Chlorpyrifos, Cypermethrin, Deltamethrin, Abamectin, Acetamiprid, Emamectin Benzoate, Imidacloprid, Indoxacarb, Lambda-Cyhalothrin, Spinosad, Thiodicarb, Gamma-Cyhalothrin, Spiromesifen, Pyridalyl, Flonicamid Flubendiamide, Triflumuron, Rynaxypyr, Beta-Cyfluthrin, Spirotetramat
o. Clothianidin, Thiamethoxam, Thiacloprid, Dinetofuran, Flubendiamide, Cyazypyr, Spinosad, Spinotoram, gamma Cyhalothrin, 4-[[(6-Chlorpyridin-3-yl)methyl] (2,2-difluorethyl)amino]furan-2(5H)-on Thiodicarb, Avermectin, Flonicamid, Pyridalyl, Spiromesifen, Sulfoxaflor
p. Cotton Fungicides: Azoxystrobin, Bixafen, Boscalid, Carbendazim, Chlorothalonil, Copper, Cyproconazole, Difenoconazole, Dimoxystrobin, Epoxiconazole, Fenamidone, Fluazinam, Fluopyram, Fluoxastrobin, Fluxapyroxad, Iprodione, Isopyrazam, Isotianil, Mancozeb, Maneb, Metominostrobin, Penthiopyrad, Picoxystrobin, Propineb, Prothioconazole, Pyraclostrobin, Quintozene, Tebuconazole, Tetraconazole, Thiophanate-methyl, Trifloxystrobin
q. Soybean Herbicides: Alachlor, Bentazone, Trifluralin, Chlorimuron-Ethyl, Cloransulam-Methyl, Fenoxaprop, Fomesafen, Fluazifop, Glyphosate, Imazamox, Imazaquin, Imazethapyr, (S-)Metolachlor, Metribuzin, Pendimethalin, Tepraloxydim, Glufosinate
r. Soybean Insecticides: Lambda-cyhalothrin, Methomyl, Imidacloprid, Clothianidin, Thiamethoxam, Thiacloprid, Acetamiprid, Dinetofuran, Flubendiamide, Rynaxypyr, Cyazypyr, Spinosad, Spinotoram, Emamectin-Benzoate, Fipronil, Ethiprole, Deltamethrin, β-Cyfluthrin, gamma and lambda Cyhalothrin, 4-[[(6-Chlorpyridin-3-yl)methyl] (2,2-difluorethyl)amino]furan-2(5H)-on, Spirotetramat, Spinodiclofen, Triflumuron, Flonicamid, Thiodicarb, beta-Cyfluthrin
s. Soybean Fungicides: Azoxystrobin, Bixafen, Boscalid, Carbendazim, Chlorothalonil, Copper, Cyproconazole, Difenoconazole, Dimoxystrobin, Epoxiconazole, Fluazinam, Fluopyram, Fluoxastrobin, Flutriafol, Fluxapyroxad, Isopyrazam, Iprodione, Isotianil, Mancozeb, Maneb, Metconazole, Metominostrobin, Myclobutanil, Penthiopyrad, Picoxystrobin, Propiconazole, Propineb, Prothioconazole, Pyraclostrobin, Tebuconazole, Tetraconazole, Thiophanate-methyl, Trifloxystrobin
t. Sugarbeet Herbicides: Chloridazon, Desmedipham, Ethofumesate, Phenmedipham, Triallate, Clopyralid, Fluazifop, Lenacil, Metamitron, Quinmerac, Cycloxydim, Triflusulfuron, Tepraloxydim, Quizalofop
u. Sugarbeet Insecticides: Imidacloprid, Clothianidin, Thiamethoxam, Thiacloprid, Acetamiprid, Dinetofuran, Deltamethrin, β-Cyfluthrin, gamma/lambda Cyhalothrin, 4-[[(6-Chlorpyridin-3-yl)methyl] (2,2-difluorethyl)amino]furan-2(5H)-on, Tefluthrin, Rynaxypyr, Cyaxypyr, Fipronil, Carbofuran
v. Canola Herbicides: Clopyralid, Diclofop, Fluazifop, Glufosinate, Glyphosate, Metazachlor, Trifluralin Ethametsulfuron, Quinmerac, Quizalofop, Clethodim, Tepraloxydim
w. Canola Fungicides: Azoxystrobin, Bixafen, Boscalid, Carbendazim, Cyproconazole, Difenoconazole, Dimoxystrobin, Epoxiconazole, Fluazinam, Fluopyram, Fluoxastrobin, Flusilazole, Fluxapyroxad, Iprodione, Isopyrazam, Mepiquat-chloride, Metconazole, Metominostrobin, Paclobutrazole, Penthiopyrad., Picoxystrobin, Prochloraz, Prothioconazole, Pyraclostrobin, Tebuconazole, Thiophanate-methyl, Trifloxystrobin, Vinclozolin
x. Canola Insecticides: Carbofuran, Thiacloprid, Deltamethrin, Imidacloprid, Clothianidin, Thiamethoxam, Acetamiprid, Dinetofuran, β-Cyfluthrin, gamma and lambda Cyhalothrin, tau-Fluvaleriate, Ethiprole, Spinosad, Spinotoram, Flubendiamide, Rynaxypyr, Cyazypyr, 4-[[(6-Chlorpyridin-3-yl)methyl] (2,2-difluorethyl)amino]furan-2(5H)-on

In particular, Brassica plants may be treated by application of at least one the compounds indicated as canola herbicides, canola fungicides or canola insecticides in the list above.

The invention additionally provides a process for producing glyphosate resistant Brassica plants and seeds thereof, comprising the step of crossing a plant consisting essentially of plant cells comprising one or two chimeric DNA molecules as herein described, with another plant or with itself, wherein the process may further comprise identifying or selecting progeny plants or seeds comprising the chimeric genes according to the invention, and/or applying an effective amount of a EPSPS inhibiting compound such as glyphosate, and harvesting seeds.

Also provided is a method for producing oil or seed meal from the Brassica plants comprising the chimeric gene or genes according to the invention, comprising the steps known in the art for extracting and processing oil from seeds of oilseedrape plant.

The invention also provides a process for increasing the glyphosate tolerance in plants, particularly Brassica plants comprising the steps of obtaining Brassica plants comprising a chimeric gene or genes as described elsewhere in the this application, and planting said Brassica plants in a field.

The following non-limiting Examples describe method and means for increasing herbicide tolerance in plants according to the invention. Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, NY and in Volumes I and 2 of Ausubel et al. (1994) Current Protocols in Molecular Biology, Current Protocols, USA. Standard materials and methods for plant molecular work are described in Plant Molecular Biology Labfax (1993) by R.D.D. Croy, jointly published by BIOS Scientific Publications Ltd (UK) and Blackwell Scientific Publications, UK. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR-Basics: From Background to Bench, First Edition, Springer Verlag, Germany.

Throughout the description and Examples, reference is made to the following sequences:

SEQ ID No.:1: nucleotide sequence of T-DNA of vector pTJN47
SEQ ID No.:2: nucleotide sequence of T-DNA of vector pTJN50
SEQ ID No.:3: nucleotide sequence of T-DNA of vector pTJN51
SEQ ID No.:4: nucleotide sequence of T-DNA of vector pTJN48
SEQ ID No.:5: nucleotide sequence of T-DNA of vector pTJN49
SEQ ID No.:6: nucleotide sequence of T-DNA of vector pTJN75
SEQ ID No.:7: amino acid sequence of the optimized transit peptide TPotp C-1Pc
SEQ ID No.:8: amino acid sequence of the 2mEPSPS-1 Pa
SEQ ID No.:9: nucleotide sequence of the Arabidopsis thaliana H3 gene 1 and H3 gene 2 for H3.3-like histone variant (X60429.1)
SEQ ID No.:10: nucleotide sequence of the Medicago sativa cultivar Chief histone H3.2 gene (U09458.1)

### EXAMPLES

### Example 1

### Construction of Chimeric DNA Molecules

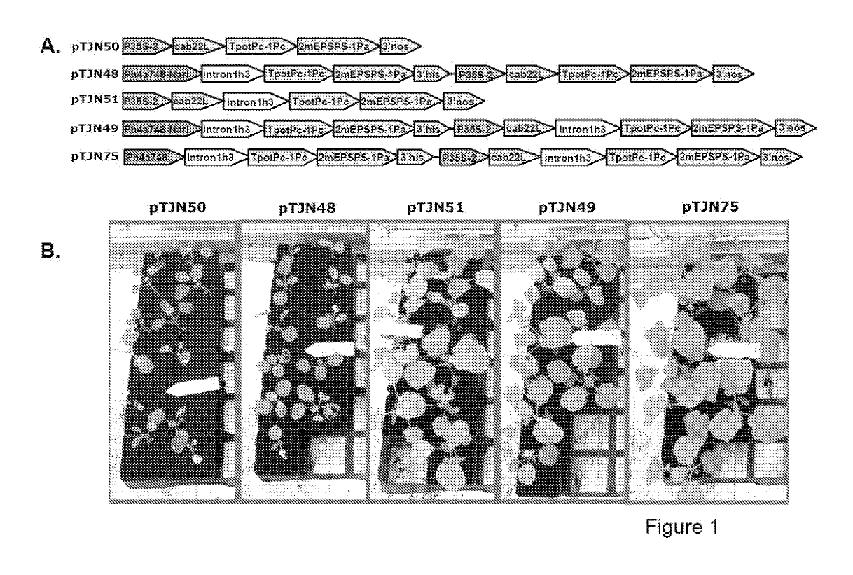

FIG. 1A provides examples of chimeric DNA molecules according to the invention. These molecules are not to be construed as the only constructs that can be assembled, but serve only as examples to those skilled in the art.

Using conventional recombinant DNA techniques the following T-DNA expression vectors were constructed (pTJN47, pTJN50, pTJN51, pTJN48, pTJN49, pTJN75) comprising the following operably linked DNA fragments:

pTJN47
a) Ph4a748-NarI: Sequence including the promoter region of the histone H4 gene of Arabidopsis thaliana (Chabouté et al., 1987, Plant Mol. Biol. 8, 179-191)
b) intron1 h3At: sequence including the first intron of gene II of the histone H3.III variant of Arabidopsis thaliana (Chaubet et al., 1992, J Mol Biol 225: 569-574)
c) TPotp C-1Pc: coding sequence of the optimized transit peptide, containing sequence of the RuBisCO small subunit genes of Zea mays (corn) and Helianthus annuus (sunflower), as described by Lebrun et al. (1996, U.S. Pat. No. 5,510,471), adapted to Brassica napus codon usage
d) 2mepsps-1 Pa: the coding sequence of the double-mutant 5-enol-pyruvylshikimate-3-phosphate synthase gene of Zea mays (corn) (Lebrun et al., 1997 WO9704103), adapted to Brassica napus codon usage
e) 3′his: sequence including the 3′ untranslated region of the histone H4 gene of Arabidopsis thaliana (Chabouté et al., 1987, supra)
   The nucleotide sequence of T-DNA of vector pTJN47 is represented in SEQ ID no. 1.

pTJN50
a) P35S2: sequence including the promoter region of the Cauliflower Mosaic Virus 35S transcript (Odell et al., 1985)
b) 5′cab22L: sequence including the leader sequence of the chlorophyl a/b binding protein gene from Petunia hybrida (Harpster et al., 1988)
c) TPotp C-1Pc: coding sequence of the optimized transit peptide, containing sequence of the RuBisCO small subunit genes of Zea mays (corn) and Helianthus annuus (sunflower), as described by Lebrun et al. (1996), adapted to Brassica napus codon usage
d) 2mepsps-1 Pa: the coding sequence of the double-mutant 5-enol-pyruvylshikimate-3-phosphate synthase gene of Zea mays (corn) (Lebrun et al., 1997), adapted to Brassica napus codon usage
e) 3′ nos: sequence including the 3′ untranslated region of the nopaline synthase gene from the T-DNA of pTiT37 (Depicker et al., 1982)
   The nucleotide sequence of T-DNA of vector pTJN50 is represented in SEQ ID no. 2.

pTJN48
a) Ph4a748-NarI: Sequence including the promoter region of the histone H4 gene of Arabidopsis thaliana (Chabouté et al., 1987)
b) intron1 h3At: sequence including the first intron of gene II of the histone H3.III variant of Arabidopsis thaliana (Chaubet et al., 1992)
c) TPotp C-1Pc: coding sequence of the optimized transit peptide, containing sequence of the RuBisCO small subunit genes of Zea mays (corn) and Helianthus annuus (sunflower), as described by Lebrun et al. (1996), adapted to Brassica napus codon usage
d) 2mepsps-1 Pa: the coding sequence of the double-mutant 5-enol-pyruvylshikimate-3-phosphate synthase gene of Zea mays (corn) (Lebrun et al., 1997), adapted to Brassica napus codon usage
e) 3′his: sequence including the 3′ untranslated region of the histone H4 gene of Arabidopsis thaliana (Chabouté et al., 1987)
f) P35S2: sequence including the promoter region of the Cauliflower Mosaic Virus 35S transcript (Odell et al., 1985)
g) 5′cab22L: sequence including the leader sequence of the chlorophyl a/b binding protein gene from Petunia hybrida (Harpster et al., 1988)
h) TPotp C-1Pc: coding sequence of the optimized transit peptide, containing sequence of the RuBisCO small subunit genes of Zea mays (corn) and Helianthus annuus (sunflower), as described by Lebrun et al. (1996), adapted to Brassica napus codon usage
i) 2mepsps-1 Pa: the coding sequence of the double-mutant 5-enol-pyruvylshikimate-3-phosphate synthase gene of Zea mays (corn) (Lebrun et al., 1997), adapted to Brassica napus codon usage
j) 3′ nos: sequence including the 3′ untranslated region of the nopaline synthase gene from the T-DNA of pTiT37 (Depicker et al., 1982)
   The nucleotide sequence of T-DNA of vector pTJN48 is represented in SEQ ID no. 3.

pTJN51
a) P35S2: sequence including the promoter region of the Cauliflower Mosaic Virus 35S transcript (Odell et al., 1985)
b) 5′cab22L: sequence including the leader sequence of the chlorophyl a/b binding protein gene from Petunia hybrida (Harpster et al., 1988)
c) intron1 h3At: sequence including the first intron of gene II of the histone H3.III variant of Arabidopsis thaliana (Chaubet et al., 1992)
d) TPotp C-1Pc: coding sequence of the optimized transit peptide, containing sequence of the RuBisCO small subunit genes of Zea mays (corn) and Helianthus annuus (sunflower), as described by Lebrun et al. (1996), adapted to Brassica napus codon usage
e) 2mepsps-1 Pa: the coding sequence of the double-mutant 5-enol-pyruvylshikimate-3-phosphate synthase gene of Zea mays (corn) (Lebrun et al., 1997), adapted to Brassica napus codon usage
f) 3′ nos: sequence including the 3′ untranslated region of the nopaline synthase gene from the T-DNA of pTiT37 (Depicker et al., 1982)
   The nucleotide sequence of T-DNA of vector pTJN51 is represented in SEQ ID no. 4.

pTJN49
a) Ph4a748-NarI: Sequence including the promoter region of the histone H4 gene of Arabidopsis thaliana (Chabouté et al., 1987)
b) intron1 h3At: sequence including the first intron of gene II of the histone H3.III variant of Arabidopsis thaliana (Chaubet et al., 1992)
c) TPotp C-1Pc: coding sequence of the optimized transit peptide, containing sequence of the RuBisCO small subunit genes of Zea mays (corn) and Helianthus annuus (sunflower), as described by Lebrun et al. (1996), adapted to Brassica napus codon usage
d) 2mepsps-1 Pa: the coding sequence of the double-mutant 5-enol-pyruvylshikimate-3-phosphate synthase gene of Zea mays (corn) (Lebrun et al., 1997), adapted to Brassica napus codon usage
e) 3′ his: sequence including the 3′ untranslated region of the histone H4 gene of Arabidopsis thaliana (Chabouté et al., 1987)
f) P35S2: sequence including the promoter region of the Cauliflower Mosaic Virus 35S transcript (Odell et al., 1985)
g) 5′cab22L: sequence including the leader sequence of the chlorophyl a/b binding protein gene from Petunia hybrida (Harpster et al., 1988, supra)
h) intron1 h3At: sequence including the first intron of gene II of the histone H3.III variant of Arabidopsis thaliana (Chaubet et al., 1992)
i) TPotp C-1Pc: coding sequence of the optimized transit peptide, containing sequence of the RuBisCO small subunit genes of Zea mays (corn) and Helianthus annuus (sunflower), as described by Lebrun et al. (1996), adapted to Brassica napus codon usage
j) 2mepsps-1 Pa: the coding sequence of the double-mutant 5-enol-pyruvylshikimate-3-phosphate synthase gene of Zea mays (corn) (Lebrun et al., 1997), adapted to Brassica napus codon usage
k) 3′nos: sequence including the 3′ untranslated region of the nopaline synthase gene from the T-DNA of pTiT37 (Depicker et al., 1982)
   The nucleotide sequence of T-DNA of vector pTJN49 is represented in SEQ ID no. 5.

pTJN75
a) Ph4a748: Sequence including the promoter region of the histone H4 gene of Arabidopsis thaliana (Chabouté et al., 1987)
b) intron1 h3At: sequence including the first intron of gene II of the histone H3.III variant of Arabidopsis thaliana (Chaubet et al., 1992)
c) TPotp C-1Pc: coding sequence of the optimized transit peptide, containing sequence of the RuBisCO small subunit genes of Zea mays (corn) and Helianthus annuus (sunflower), as described by Lebrun et al. (1996), adapted to Brassica napus codon usage
d) 2mepsps-1 Pa: the coding sequence of the double-mutant 5-enol-pyruvylshikimate-3-phosphate synthase gene of Zea mays (corn) (Lebrun et al., 1997), adapted to Brassica napus codon usage
e) 3′his: sequence including the 3′ untranslated region of the histone H4 gene of Arabidopsis thaliana (Chabouté et al., 1987)
f) P35S2: sequence including the promoter region of the Cauliflower Mosaic Virus 35S transcript (Odell et al., 1985)
g) 5′cab22L: sequence including the leader sequence of the chlorophyl a/b binding protein gene from Petunia hybrida (Harpster et al., 1988)
h) intron1 h3At: sequence including the first intron of gene II of the histone H3.III variant of Arabidopsis thaliana (Chaubet et al., 1992)
i) TPotp C-1Pc: coding sequence of the optimized transit peptide, containing sequence of the RuBisCO small subunit genes of Zea mays (corn) and Helianthus annuus (sunflower), as described by Lebrun et al. (1996), adapted to Brassica napus codon usage
j) 2mepsps-1 Pa: the coding sequence of the double-mutant 5-enol-pyruvylshikimate-3-phosphate synthase gene of Zea mays (corn) (Lebrun et al., 1997), adapted to Brassica napus codon usage
k) 3′ nos: sequence including the 3′ untranslated region of the nopaline synthase gene from the T-DNA of pTiT37 (Depicker et al., 1982)
   The nucleotide sequence of T-DNA of vector pTJN75 is represented in SEQ ID no. 6.

Codon optimization for Brassica napus was performed using Leto 1.0 gene optimizing software (Entelechon GmbH, Germany)

### Example 2

### Agrobacterium-Mediated Transformation of Brassica napus with the T-DNA Vectors of Example 1

The resulting T-DNA vectors were introduced in Agrobacterium tumefaciens C58C1R1f(pGV4000) and transformants were selected using spectinomycin and streptomycin according to methods known in the art.

The Agrobacterium strains were used to transform the Brassica napus var. PPS02-144B according to methods known in the art and transgenic plants were selected for glyphosate tolerance (0.4 kg a.i./ha) and verified for single copy number using Southern blotting and RT-PCR. TO plants were backcrossed with wild type plants and the resulting T1 generation was used for glyphosate tolerance tests in the greenhouse.

### Example 3

### Measurement of Glyphosate Tolerance

To analyze glyphosate tolerance, for each transformation event 51 T1 seeds were sown in a greenhouse, and treatment post-emergence at the 2-4 leaf stage was carried out with a dose of glyphosate of 2.0 kg a.i./ha, corresponding to 5× the conventional dose used in the greenhouse. Ten days after spraying, photographs of surviving plants of one representative event per construct were taken (FIG. 1A) and the surviving populations were scored for the following parameters:

For assessment of vigor, plants were evaluated on a scale of 1 to 9, where 1=dead, 3=poor, 6=some aberrant phenotype and 9=vigorous. The average values (Av) and standard deviations (sd) of 5 representative events per construct are represented in Table 1.

For assessment of PPTOX, plants were evaluated on a scale of 1 to 9, where 1=completely yellowing, 5=50% of plant is yellow and 9=no yellowing. The average values (Av) and standard deviations (sd) of 5 representative events per construct are represented in Table 1.

TABLE 1

| | Vigor | PPTOX |
|---|---|---|
| | Av (sd) | Av (sd) |
| pTJN50 | 1.2 (0.4) | 6.0 (0.0) |
| pTJN48 | 1.2 (0.4) | 5.2 (0.4) |
| pTJN51 | 5.0 (0.0) | 5.6 (0.5) |
| pTJN49 | 5.4 (0.5) | 6.8 (0.4) |
| pTJN75 | 7.0 (0.0) | 7.6 (0.5) |

When comparing the appearance of the plants as depicted in figure FIG. 1B with the values of Table 1, the vigor measurements appear to correlate best to the level of glyphosate tolerance. pTJN51 plants having the chimeric DNA molecule containing 2mEPSPS under control of the P35S2 promoter with intron1 h3 scored significantly better on vigor than similar pTJN50 plants without the intron1 h3. A significantly higher vigor score upon introduction of the intron1 h3 was also observed when comparing pTJN49 plants comprising the P35S2 promoter with intron1 h3 to pTJN48 plants that lack the intron1 h3. Introduction of a second chimeric DNA molecule with 2mEPSPS under the control of the truncated pH4a748-NarI promoter with intron1 h3 did not increase vigor of pTJN48 plants or pTJN49 plants when compared to plants that lack this additional molecule, pTJN50 and pTJN51 respectively. Surprisingly, pTJN75 plants having a second chimeric DNA molecule comprising the full length pH4a748 promoter with intron1 h3 in addition to the chimeric DNA molecule comprising P35S2 with intron1 h3 displayed higher vigor when compared to pTJN51 plants without the second chimeric DNA molecule, and also when compared to similar plants with the truncated pH4a748-NarI promoter (pTJN49). Of note, pTJN47 plants having only a chimeric DNA molecule with 2mEPSPS under the control of the truncated pH4a748-NarI promoter and intron1 h3 did not provide seed when primary transformants were sprayed with 0.4 kg a.i./ha glyphosate, indicating that this truncated pH4a748-NarI promoter-intron1 h3 combination does not induce sufficient EPSPS expression to tolerate the applied glyphosate dosage.

Previous similar experiments with a non-codon-optimized 2mEPSPS resulted in plants with limited glyphosate tolerance, with vigor scores of at most 4.7 (0.5) after spraying with 2×0.4 kg a.i./ha glyphosate.

These data thus clearly show the improvement offered by the use of replacement histone H3 introns in combination with the constitutive 35S promoter and the full length H4a748 promoter to drive quantitative and qualitative expression of a glyphosate tolerant EPSPS in order to obtain plants with increased glyphosate tolerance.

### Example 4

### Construction of Further Chimeric DNA Molecules

Using conventional recombinant DNA techniques, the following T-DNA expression vectors were constructed by operably linking the following DNA fragments:

pTJR2
a) Ph4a-748-NarI: Sequence including the promoter region of the histone H4 gene of Arabidopsis thaliana (Chabouté et al., 1987)
b) TPotp C-1Pc: coding sequence of the optimized transit peptide, containing sequences of RuBisCO small subunit genes of Zea mays (corn) and Heliantus annuus (sunflower), as described by Lebrun et al. (1996)
c) 2mepsps: coding sequence of the double mutant 5-enol-pyruvylshilimte-3-phosphate synthase gene of Zea mays (corn) (Lebrun et al., 1997)
d) 3′his: sequence including the 3′ untranslated region of the histone H4 gene of Arabidopsis thaliana (Chabouté et al., 1987)

pTJN73
a) Ph4a-748: Sequence including the promoter region of the histone H4 gene of Arabidopsis thaliana (Chabouté et al., 1987)
b) TPotp C-1Pc: coding sequence of the optimized transit peptide, containing sequences of RuBisCO small subunit genes of Zea mays (corn) and Heliantus annuus (sunflower), as described by Lebrun et al. (1996)
c) 2mepsps-1 Pa: coding sequence of the double mutant 5-enol-pyruvylshilimte-3-phosphate synthase gene of Zea mays (corn) (Lebrun et al., 1997), adapted to Brassica napus codon usage
d) 3′his: sequence including the 3′ untranslated region of the histone H4 gene of Arabidopsis thaliana (Chabouté et al., 1987)

pTEM2
a) Ph4a-748: Sequence including the promoter region of the histone H4 gene of Arabidopsis thaliana (Chabouté et al., 1987)
b) TPotp C-1Pc: coding sequence of the optimized transit peptide, containing sequences of RuBisCO small subunit genes of Zea mays (corn) and Heliantus annuus (sunflower), as described by Lebrun et al. (1996)
c) 2mepsps: coding sequence of the double mutant 5-enol-pyruvylshikimate-3-phosphate synthase gene of Zea mays (corn) (Lebrun et al., 1997)
d) 3′his: sequence including the 3′ untranslated region of the histone H4 gene of Arabidopsis thaliana (Chabouté et al., 1987)

### Example 5

### Comparison of the Transformation Efficiency of Different T-DNA Vectors

T-DNA vectors comprising either the short promoter region of the histone H4 gene or the long version and further comprising either the coding sequence of the double mutant 5-enol-pyruvylshikimate-3-phosphate synthase gene of Zea mays (corn) or the coding encoding double mutant 5-enol-pyruvylshikimate-3-phosphate synthase gene of Zea mays adapted to Brassica napus codon usage (pTJN47, pTJR2, pTJN73 or pTEM2) were used to transform Brassica napus protoplasts through co-cultivation with Agrobacteria comprising these respective T-DNA vectors. Three independent experiments were performed with each vector (10 selection plates for each experiment). In the case of pTJN47 only 2 independent experiments were performed. The number of transformed colonies was counted after 3 weeks of selection on 0.25 mM Glyphosate.

| | | |
|---|---|---|
| pTJN47 | experiment 1 | 312 colonies |
| pTJN47 | experiment 2 | 144 colonies |
| | | Average = 228 (n = 20) |
| pTJR2 | experiment 1 | 237 colonies |
| pTJR2 | experiment 2 | 172 colonies |
| pTJR2 | experiment 3 | 105 colonies |
| | | Average = 171 (n = 30) |
| pTEM2 | experiment 1 | 566 colonies |
| pTEM2 | experiment 2 | 428 colonies |
| pTEM2 | experiment 3 | 860 colonies |
| | | Average = 618 (n = 30) |
| pTJN73 | experiment 1 | 990 colonies |
| pTJN73 | experiment 2 | 53 colonies |
| pTJN73 | experiment 3 | 940 colonies |
| | | Average = 828 (n = 30) |

A clear difference in transformation efficiency can be observed between vectors containing the long histone promoter versus vectors containing the short histone promoter.

### Example 6

### Field Trials

The 4 best events selected for pTJN49 and pTJN75 transformed Brassica napus lines were submitted to a field trial.

| Construct | RATE | Events | PPTOX | VIG_BH | VIG_AH | VIG_AH2 | VIG_AH3 |
|---|---|---|---|---|---|---|---|
| pTJN49 | 1 APP 2000g ai | GLBN0002-06001 | 5.0 | 7.0 | 6.5 | 8.0 | 8.0 |
| | 1 APP 2000g ai | GLBN0002-06501 | 4.5 | 7.5 | 7.0 | 8.0 | 8.0 |
| | 1 APP 2000g ai | GLBN0002-08101 | 4.7 | 7.7 | 6.7 | 7.7 | 7.7 |
| | 1 APP 2000g ai | GLBN0002-08301 | 5.3 | 7.7 | 7.0 | 8.3 | 8.0 |
| | | average | 4.9 | 7.5 | 6.8 | 8.0 | 7.9 |
| pTJN75 | 1 APP 2000g ai | GLBN0033-07301 | 5.5 | 6.5 | 7.0 | 8.5 | 8.0 |
| | 1 APP 2000g ai | GLBN0033-10201 | 8.0 | 8.0 | 7.7 | 9.0 | 8.0 |
| | 1 APP 2000g ai | GLBN0037-03401 | 6.7 | 7.3 | 8.0 | 9.0 | 8.0 |
| | 1 APP 2000g ai | GLBN0037-03801 | 8.0 | 7.0 | 7.5 | 8.5 | 8.5 |
| | | average | 7.0 | 7.2 | 7.5 | 8.8 | 8.1 |
| | | p | 0.0132 | 0.4673 | 0.0216 | 0.0074 | 0.2192 |
| | | mean (pTJN49-pTJN75) | −2.175 | 0.275 | −0.75 | −0.75 | −0.200 |
| | | 95% Int | −3.706 to −0.644 | −0.592 to | −1.345 to −0.155 | −1.213 to −0.287 | −0.557 to |
| | | | | 1.142 | | | 0.157 |
| | | t | 3.4763 | 0.7759 | 3.0833 | 3.962 | 1.372 |
| | | df | 6 | 6 | 6 | 6 | 6 |
| | | SE | 0.626 | 0.354 | 0.243 | 0.189 | 0.146 |
| | | Significance | *** | ns | *** | **** | ns |

RCBD design, split block, 3 repetitions, single row plots- 1 application and 2 applications of glyphosate
PPTOX: phytotoxicity rating;
VIG_BH: vigor before herbicide application;
VIG_AH, VIG_AH2 and VIG_AH3: vigor 7, 14 and 21 days after herbicide application, respectively.
Statistical analysis: two-tailed unpaired t test.
***: very significantly different;
****: extremely significantly different;
ns: not significantly different

| Construct | RATE | Events | PPTOX | VIG_BH | VIG_AH | VIG_AH2 | VIG_AH3 |
|---|---|---|---|---|---|---|---|
| pTJN49 | 2 APP 2000g ai | GLBN0002-06001 | 5.0 | 7.0 | 6.5 | 6.5 | 6.5 |
| | 2 APP 2000g ai | GLBN0002-06501 | 5.0 | 7.5 | 7.0 | 7.0 | 6.5 |
| | 2 APP 2000g ai | GLBN0002-08101 | 4.3 | 7.3 | 6.0 | 7.0 | 6.7 |
| | 2 APP 2000g ai | GLBN0002-08301 | 5.0 | 7.3 | 7.0 | 6.0 | 6.0 |
| | | Average | 4.8 | 7.3 | 6.6 | 6.6 | 6.4 |
| pTJN75 | 2 APP 2000g ai | GLBN0033-07301 | 7.5 | 6.5 | 7.0 | 8.0 | 7.0 |
| | 2 APP 2000g ai | GLBN0033-10201 | 7.0 | 7.7 | 8.0 | 9.0 | 7.7 |
| | 2 APP 2000g ai | GLBN0037-03401 | 7.3 | 7.3 | 7.7 | 8.3 | 7.3 |
| | 2 APP 2000g ai | GLBN0037-03801 | 7.5 | 7.5 | 7.5 | 8.0 | 7.5 |
| | | Average | 7.3 | 7.3 | 7.5 | 8.3 | 7.4 |
| | | p | 0.0001 | 0.9324 | 0.0272 | 0.0023 | 0.0041 |
| | | mean | −0.25 | 0.025 | −0.925 | −1.700 | −0.950 |
| | | (pTJN49-pTJN75) | | | | | |
| | | 95% Int | −3.017 to −1.983 | −0.666 to | −1.704 to −0.146 | −2.522 to −0.878 | −1.467 to −0.433 |
| | | | | 0.716 | | | |
| | | t | 11.84 | 0.0885 | 2.904 | 5.0591 | 4.4992 |
| | | df | 6 | 6 | 6 | 6 | 6 |
| | | SE | 0.211 | 0.282 | 0.319 | 0.336 | 0.211 |
| | | Significance | **** | ns | *** | **** | **** |

Different embodiments of the invention can thus be summarized as in the following paragraphs
Paragraph 1. A plant comprising a chimeric DNA molecule comprising the following operably linked DNA fragments:
   a) a plant-expressible constitutive promoter;
   b) a DNA region encoding a 5′UTR;
   c) a DNA region encoding an intron 1 of a plant replacement histone gene;
   d) a DNA region encoding a transit peptide;
   e) a DNA region encoding a glyphosate-tolerant 5-enolpyruvylshikimate-3-phosphate synthase (EPSPS); and
   f) a 3′ transcription termination and polyadenylation region, functional in a plant
Paragraph 2. A plant according to paragraph 1, wherein the constitutive promoter is the CaMV 35S promoter.
Paragraph 3. A plant according to paragraph 1 or 2, wherein the constitutive promoter comprises nt 2352 to 2770 of SEQ ID No.: 2.
Paragraph 4. A plant according to any one of paragraphs 1-3, wherein the intron 1 comprises a nucleotide sequence selected from the group consisting of genbank accession number X60429.1 or U09458.1.
Paragraph 5. A plant according to any one of paragraphs 1-4, wherein the intron 1 comprises nt 692-1100 or nt 2984-3064 of SEQ ID no. 9 or nt 555 to 668 of SEQ ID no. 10.
Paragraph 6. A plant according to any one of paragraphs 1-5, wherein the glyphosate-tolerant EPSPS encoding DNA region comprises the nucleotide sequence of the 2mEPSPS gene of Zea mays.
Paragraph 7. A plant according to any one of paragraphs 1-5, wherein the glyphosate-tolerant EPSPS encoding DNA region encodes the amino acid sequence of SEQ ID no. 8.
Paragraph 8. A plant according to paragraph 7, wherein the glyphosate-tolerant EPSPS encoding DNA region is adapted to Brassica napus codon usage.
Paragraph 9. A plant according to paragraph 7 or 8, wherein the glyphosate-tolerant EPSPS encoding DNA region comprises nt 997-2334 of SEQ ID no. 1.
Paragraph 10. A plant according to any one of paragraphs 1-9, wherein the 5′UTR comprises the leader sequence of the chlorophyl a/b binding protein gene from Petunia hybrida.
Paragraph 11. A plant according to paragraph 10, wherein said 5′UTR encoding DNA region comprises nt 2283-2351 of SEQ ID no. 2.
Paragraph 12. A plant according to any one of paragraphs 1-11, wherein the transit peptide encoding DNA region comprises sequences of the RuBisCO small subunit genes of Zea mays and Helianthus annuus.
Paragraph 13. A plant according to any one of paragraph 1-11, wherein the transit peptide encoding DNA region encodes the aminoacid sequence of SEQ ID no. 7.
Paragraph 14. A plant according to paragraph 13, wherein the transit peptide encoding DNA region is adapted to Brassica napus codon usage.
Paragraph 15. A plant according to paragraph 13 or 14, wherein the transit peptide encoding DNA region comprises nt 2335-2706 of SEQ ID no. 1.
Paragraph 16. A plant according to any one of paragraphs 1-15, wherein the 3′ transcription termination and polyadenylation region comprises nt 307-572 or nt 3252-3966 of SEQ ID no. 7.
Paragraph 17. A plant according to any one of paragraphs 1-16, further comprising a second chimeric DNA molecule, the second chimeric DNA molecule comprising the following operably linked DNA fragments:
   a) a promoter sequence of the histone H4 gene of Arabidopsis thaliana;
   b) a DNA region encoding an intron 1 of a plant replacement histone gene;
   c) a DNA region encoding a transit peptide;
   d) a DNA region encoding a glyphosate-tolerant EPSPS; and
   e) a 3′ transcription termination and polyadenylation region.
Paragraph 18. A plant according to paragraph 17, wherein the histone H4 promoter sequence comprises nt 6166-7087 of SEQ ID no. 6.
Paragraph 19. A plant according to paragraph 17 or 18, wherein the intron 1 comprises a nucleotide sequence selected from the group consisting of genbank accession number X60429.1 or U09458.1.
Paragraph 20. A plant according to any one of paragraphs 17-19, wherein the intron one comprises nt 692-1100 or nt 2984-3064 of SEQ ID no. 9 or nt 555 to 668 of SEQ ID no. 10.
Paragraph 21. A plant according to any one of paragraphs 17-20, wherein the glyphosate-tolerant EPSPS encoding DNA region comprises the coding sequence of the dmEPSPS gene of Zea mays.
Paragraph 22. A plant according to any one of paragraphs 17-20, wherein the glyphosate-tolerant EPSPS encoding DNA region encodes the amino acid sequence of SEQ ID no. 8.
Paragraph 23. A plant according to paragraph 22, wherein the glyphosate-tolerant EPSPS encoding DNA region is adapted to Brassica napus codon usage.
Paragraph 24. A plant according to paragraph 22 or 23, wherein the glyphosate-tolerant EPSPS encoding DNA region comprises nt 997-2334 of SEQ ID no. 1.
Paragraph 25. A plant according to any one of paragraphs 17-24, wherein the transit peptide encoding sequence comprises sequences of the RuBisCO small subunit genes of Zea mays and Helianthus annuus.
Paragraph 26. A plant according to any one of paragraphs 17 to 24, wherein the transit peptide encoding DNA region encodes the aminoacid sequence of SEQ ID no. 7.
Paragraph 27. A plant according to paragraph 26, wherein the transit peptide encoding DNA region is adapted to Brassica napus codon usage.
Paragraph 28. A plant according to paragraph 26 or 27, wherein the transit peptide encoding DNA region comprises nt 2335-2706 of SEQ ID no. 1.
Paragraph 29. A plant according to any one of paragraphs 17-28, wherein the 3′ transcription termination and polyadenylation region comprises nt 307-572 or nt 3252-3966 of SEQ ID no. 7.
Paragraph 30. The plant of any one of paragraphs 1 to 29 which is a Brassica plant.
Paragraph 31. The plant of any one of paragraphs 1-30 which is oilseed rape.
Paragraph 32. The plant of any one of paragraphs 1 to 31 which is Brassica napus, Brassica rapa, Brassica campestris or Brassica juncea.
Paragraph 33. A plant cell of the plant of any one of paragraphs 1-32 comprising the chimeric genes as described in any of paragraphs 1-29.
Paragraph 34. A seed of the plant of any one of paragraphs 1-32 comprising the chimeric genes as described in any of paragraphs 1-29.
Paragraph 35. A chimeric DNA molecule as described in any one of paragraphs 1-29.
Paragraph 36. A cloning and/or expression vector for transforming plants, comprising at least one of the chimeric DNA molecules of paragraph 35.
Paragraph 37. A method for treating plants as described in any one of paragraphs 1-32, characterized in that the plants are treated with EPSPS-inhibiting herbicide.
Paragraph 38. A method according to paragraph 37, wherein the EPSPS-inhibiting herbicide is glyphosate.
Paragraph 39. A method according to paragraph 38, wherein the plant is tolerant to an application of at least 2.0 kg/ha.
Paragraph 40. Use of a chimeric DNA molecule according to paragraph 35 to generate a glyphosate tolerant plant.

1. A plant cell or a plant comprising a chimeric DNA molecule comprising the following operably linked DNA fragments:
a) a plant-expressible constitutive promoter;
b) a DNA region encoding a 5′UTR;
c) a DNA region encoding an intron 1 of a plant replacement histone gene;
d) a DNA region encoding a transit peptide;
e) a DNA region encoding a glyphosate-tolerant 5-enolpyruvylshikimate-3-phosphate synthase (EPSPS); and
a 3′ transcription termination and polyadenylation region functional in a plant cell.

2. The plant cell or a plant according to claim 1, wherein said constitutive promoter is the CaMV 35S promoter.

3. The plant cell or a plant according to any one of claim 1, wherein said intron 1 comprises nt 692-1100 or nt 2984-3064 of SEQ ID no. 9 or nt 555 to 668 of SEQ ID no. 10.

4. The plant cell or a plant according to claim 1, wherein said glyphosate-tolerant EPSPS encoding DNA region encodes the amino acid sequence of SEQ ID no. 8.

5. The plant cell or a plant according to claim 4, wherein said glyphosate-tolerant EPSPS encoding DNA region comprises nt 997-2334 of SEQ ID no. 1.

6. The plant cell or a plant according to claim 1, further comprising a second chimeric DNA molecule, said second chimeric DNA molecule comprising the following operably linked DNA fragments:
a) a promoter sequence of the histone H4 gene of Arabidopsis thaliana;
b) a second DNA region encoding an intron 1 of a plant replacement histone gene;
c) a second DNA region encoding a transit peptide;
d) a second DNA region encoding a glyphosate-tolerant EPSPS; and
e) a second 3′ transcription termination and polyadenylation region functional in a plant cell.

7. The plant cell or a plant according to claim 6, wherein said histone H4 promoter sequence comprises nt 6166-7087 of SEQ ID no. 6.

8. The plant cell or a plant according to claim 1, wherein said intron 1 comprises nt 692-1100 or nt 2984-3064 of SEQ ID no. 9 or nt 555 to 668 of SEQ ID no. 10.

9. The plant cell or a plant according to claim 1, wherein said glyphosate-tolerant EPSPS encoding DNA region encodes the amino acid sequence of SEQ ID no. 8.

10. The plant cell or a plant according to claim 9, wherein said glyphosate-tolerant EPSPS encoding DNA region comprises nt 997-2334 of SEQ ID no. 1.

11. The plant cell or a plant of any one of claims 1 to 10 which is a Brassica plant.

12. The plant of any one of claims 1-10 which is oilseed rape.

13. A seed of a plant of any one of claims 1-10.

14. A chimeric DNA molecule as described in any one of claims 1-10.

15. A method for growing plants in the field, comprising growing plants as described in any one of claims 1-10 and treating said plants with an EPSPS-inhibiting herbicide.

16. Use of a chimeric DNA molecule according to claim 14 to generate a glyphosate tolerant plant.


SEQUENCE LISTING (10 sequences)

SEQ ID NO: 1 - DNA, 3370 bp, Artificial
  nucleotide sequence of T-DNA of pTJN47
aattacaacggtatatatcctgccagtactcggccgtcgaccgcggtaccccggaattaagcttgcatgcctgcaggtttaaacagtcgactctagacttaattaaggatccggcgcgccgcatgccccgatcaaatctgagggacgttaaagcgatgataaattggaaccagaatatagaatctttgttctgctctagcttttcttctgtacattttttacgattagactatgattttcattcaataaccaaaattctgaagtttgtcatcaagttgctcaatcaaacttgtaccggtttgtttcggttttatatcagctcactgttacactttaaccaaaatcggtttatgtcttaataaaggaattgagtcggtttaactcatatccgtaccaatgcgacgtcgtgtccgcgtttcagtagctttgctcattgtcttctacgggaactttcccggacataggaaccgccctttcgttatcctcatccatcgtgaaatcaggaaataaatgttcgaagatttgaggtcaaaagtcgaatttcatgttgtctcttctatttagatacaaaattgaagcaattttcaccaatttaatgccaaaatttaaaacaacgctgataaagtgaaacttgattcgatttatatttcaaccgaaactgctgaagcaagaagaaaaagcgtaattacacataacaagaacgctaccgcaaactactaaacgccaaacccaatacaaaagtaaaacgcagacgcttaagtgagaaacccagaaaacacaaacgcggatcggggctagcttagttcttcacgaacgtcgagagaacatcgaagtagtctgggaaagtctttctcgtgcaaccagggtctctgattgtaactggaacctcagcacatgcagcaagagagaatgccatagccatccgatgatcatcgtaggtatcgattgccgtaacgttgagcttttccggaggcgtaatgatgcagtaatccggtccttcctcaactgaagctccgagcttcgtaagctctgttctgatggcaaccattcgttcagtctccttgactctccacgaagcaacatctctgatagctgtcgggccatcggcaaacaaagcgacaacagcaagtgtcatggcaacgtctggcatcttgttcatgttcacgtcgatagccttgaggtgctttcttccaaatggctctctaggtggacctgtaacagtgactgaagtctctgtccaggtaacctttgcacccatcatctcaagaacctcagcgaacttgacatcaccttgaagactggtcgttccacaaccttcgactgtaactgttcctccagtgatagctgcaccagcaaggaagtaactagcagatgacgcatctccttcaacgtaagcgttcttcggactcttgtacttctgtcctcccttgatgtagaatctgtcccagctgtcagaatgttctgccttaactccgaatcgctccataagacgaagggtcatctcaacgtaggggatagagatgagcttgtcgatgatctcgatctcaacgtcacctaaagcaagtggtgcagccataagtagagcagacaagtactgactggagatggatccgctaagtttaacctttccacctggaagtcctccaattccgttgactctaacaggtgggcaatcagttccaaggaagcaatcaacatccgctcctagctgcttcaatccaacaacaaggtcaccgattggtctctctctcattctaggcacgccatcaagaacgtaagtggcatttcctccagcagcagtaacagcagcagtaagagatctcatggcgattcctgcgttaccgaggaaaagttgaacctcctccttggcatcttcaacagggaactttccaccacatcccacaacaacagctctctttgcagccttatccgcttcaacagaaagcccaagtgttctcaaggcacctagcatgtagtgaacgtcttcggagtttaggaggttgtcaacaactgtagtcccctcagatagagctgcaagaagcaagatgcggttggaaagagacttagacccaggtagcttaacggtaccgcttatctccttgatgggttggagaacgatctcttcagcacctgccatacatctgattctgcctccgtttgacacgttcccaagagatctggaactccttcttgcaaccggaagtgatgctgtagacttgagtccttggaatggagcaacagcagtagcagaagacgccatcataactgtcggagccatagacaaaggaggcaagtatgagagggtctcgaacttcttgttgccatatgctggccaaacttgcatacactgaactctacctccgttagagggtagggtactgaaatcgttagccttcttagttgtggggaaagctgcattggacttaagaccggtgaatggagcaaccatgttagcttgtgcaggtgcagttcttgaaaccgtcgcaacagatgagctgatggaagccatggttttggatctgcgcatttaacaagaaattgaacagtcaattggggattttcattatccataactaaattttgaagaaattggaatactaaacgtcaccacttaaaaccctaatccagatgaatcgttatcgaaccagatataaccaaaaggggcaaaattgactcgaaaaccctagttctcgatacacggctaggtaatgacaatcgcacacagacaaatctggttatacagaacttcgaagcaagaaaaaaacgatgaagaatggatcatccaataaatcgactagactcaatcttcacaggtttatcgatccagcaaacttaaaagacggacctttattttcaaactggaatgggacaaaacccgaaactctattgtcgtaaaatcagatcgcggagacagtaacagaaaaaacattaaaaagtaatggaaagacctaaacccctgatctaattacaaacaaatcatacctgttcttcgcctgagtacgtatcgagagaaattgatgtctgtagaagaagaagaacggttaagagtagatttgggtgagaaagatgtgaaattgtttttataggcaaagacggagagtctattttttgagcaatcagatcgcatattaaatctaacggctgagatatcgatccgtgtgtacaataaaatgatgtataaaccgtcgatctgttttaatcgacggttcatattagtgatccgcgtgatggcagtgatagccactaagaatcgtcttttgttttacatgtggcgcctagggcgatcgccctcgaggcattacggcattacggcactcgcgagggtcccaattcgagcatggagccatttacaattgaatatatcctgccg

SEQ ID NO: 2 - DNA, 2668 bp, Artificial
  nucleotide sequence of T-DNA of pTJN50
aattacaacggtatatatcctgccagtactcggccgtcgaccgcggtaccccggaattaagcttgcatgcctgcaggattaccctgttatccctatggcgcgccgcatgcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcgctagcttagttcttcacgaacgtcgagagaacatcgaagtagtctgggaaagtctttctcgtgcaaccagggtctctgattgtaactggaacctcagcacatgcagcaagagagaatgccatagccatccgatgatcatcgtaggtatcgattgccgtaacgttgagcttttccggaggcgtaatgatgcagtaatccggtccttcctcaactgaagctccgagcttcgtaagctctgttctgatggcaaccattcgttcagtctccttgactctccacgaagcaacatctctgatagctgtcgggccatcggcaaacaaagcgacaacagcaagtgtcatggcaacgtctggcatcttgttcatgttcacgtcgatagccttgaggtgctttcttccaaatggctctctaggtggacctgtaacagtgactgaagtctctgtccaggtaacctttgcacccatcatctcaagaacctcagcgaacttgacatcaccttgaagactggtcgttccacaaccttcgactgtaactgttcctccagtgatagctgcaccagcaaggaagtaactagcagatgacgcatctccttcaacgtaagcgttcttcggactcttgtacttctgtcctcccttgatgtagaatctgtcccagctgtcagaatgttctgccttaactccgaatcgctccataagacgaagggtcatctcaacgtaggggatagagatgagcttgtcgatgatctcgatctcaacgtcacctaaagcaagtggtgcagccataagtagagcagacaagtactgactggagatggatccgctaagtttaacctttccacctggaagtcctccaattccgttgactctaacaggtgggcaatcagttccaaggaagcaatcaacatccgctcctagctgcttcaatccaacaacaaggtcaccgattggtctctctctcattctaggcacgccatcaagaacgtaagtggcatttcctccagcagcagtaacagcagcagtaagagatctcatggcgattcctgcgttaccgaggaaaagttgaacctcctccttggcatcttcaacagggaactttccaccacatcccacaacaacagctctctttgcagccttatccgcttcaacagaaagcccaagtgttctcaaggcacctagcatgtagtgaacgtcttcggagtttaggaggttgtcaacaactgtagtcccctcagatagagctgcaagaagcaagatgcggttggaaagagacttagacccaggtagcttaacggtaccgcttatctccttgatgggttggagaacgatctcttcagcacctgccatacatctgattctgcctccgtttgacacgttcccaagagatctggaactccttcttgcaaccggaagtgatgctgtagacttgagtccttggaatggagcaacagcagtagcagaagacgccatcataactgtcggagccatagacaaaggaggcaagtatgagagggtctcgaacttcttgttgccatatgctggccaaacttgcatacactgaactctacctccgttagagggtagggtactgaaatcgttagccttcttagttgtggggaaagctgcattggacttaagaccggtgaatggagcaaccatgttagcttgtgcaggtgcagttcttgaaaccgtcgcaacagatgagctgatggaagccatggttttggtttaataagaagagaaaagagttcttttgttatggctgaagtaatagagaaatgagctcgagtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcaggggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttctagggcgatcgccctcgaggcattacggcattacggcactcgcgagggtcccaattcgagcatggagccatttacaattgaatatatcctgccg

SEQ ID NO: 3 - DNA, 3150 bp, Artificial
  nucleotide sequence of T-DNA of pTJN51
aattacaacggtatatatcctgccagtactcggccgtcgaccgcggtaccccggaattaagcttgcatgcctgcaggattaccctgttatccctatggcgcgccgcatgcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcgctagcttagttcttcacgaacgtcgagagaacatcgaagtagtctgggaaagtctttctcgtgcaaccagggtctctgattgtaactggaacctcagcacatgcagcaagagagaatgccatagccatccgatgatcatcgtaggtatcgattgccgtaacgttgagcttttccggaggcgtaatgatgcagtaatccggtccttcctcaactgaagctccgagcttcgtaagctctgttctgatggcaaccattcgttcagtctccttgactctccacgaagcaacatctctgatagctgtcgggccatcggcaaacaaagcgacaacagcaagtgtcatggcaacgtctggcatcttgttcatgttcacgtcgatagccttgaggtgctttcttccaaatggctctctaggtggacctgtaacagtgactgaagtctctgtccaggtaacctttgcacccatcatctcaagaacctcagcgaacttgacatcaccttgaagactggtcgttccacaaccttcgactgtaactgttcctccagtgatagctgcaccagcaaggaagtaactagcagatgacgcatctccttcaacgtaagcgttcttcggactcttgtacttctgtcctcccttgatgtagaatctgtcccagctgtcagaatgttctgccttaactccgaatcgctccataagacgaagggtcatctcaacgtaggggatagagatgagcttgtcgatgatctcgatctcaacgtcacctaaagcaagtggtgcagccataagtagagcagacaagtactgactggagatggatccgctaagtttaacctttccacctggaagtcctccaattccgttgactctaacaggtgggcaatcagttccaaggaagcaatcaacatccgctcctagctgcttcaatccaacaacaaggtcaccgattggtctctctctcattctaggcacgccatcaagaacgtaagtggcatttcctccagcagcagtaacagcagcagtaagagatctcatggcgattcctgcgttaccgaggaaaagttgaacctcctccttggcatcttcaacagggaactttccaccacatcccacaacaacagctctctttgcagccttatccgcttcaacagaaagcccaagtgttctcaaggcacctagcatgtagtgaacgtcttcggagtttaggaggttgtcaacaactgtagtcccctcagatagagctgcaagaagcaagatgcggttggaaagagacttagacccaggtagcttaacggtaccgcttatctccttgatgggttggagaacgatctcttcagcacctgccatacatctgattctgcctccgtttgacacgttcccaagagatctggaactccttcttgcaaccggaagtgatgctgtagacttgagtccttggaatggagcaacagcagtagcagaagacgccatcataactgtcggagccatagacaaaggaggcaagtatgagagggtctcgaacttcttgttgccatatgctggccaaacttgcatacactgaactctacctccgttagagggtagggtactgaaatcgttagccttcttagttgtggggaaagctgcattggacttaagaccggtgaatggagcaaccatgttagcttgtgcaggtgcagttcttgaaaccgtcgcaacagatgagctgatggaagccatggttttggatctgcgcatttaacaagaaattgaacagtcaattggggattttcattatccataactaaattttgaagaaattggaatactaaacgtcaccacttaaaaccctaatccagatgaatcgttatcgaaccagatataaccaaaaggggcaaaattgactcgaaaaccctagttctcgatacacggctaggtaatgacaatcgcacacagacaaatctggttatacagaacttcgaagcaagaaaaaaacgatgaagaatggatcatccaataaatcgactagactcaatcttcacaggtttatcgatccagcaaacttaaaagacggacctttattttcaaactggaatgggacaaaacccgaaactctattgtcgtaaaatcagatcgcggagacagtaacagaaaaaacattaaaaagtaatggaaagacctaaacccctgatctaattacaaacaaatcatacctgttcttcgcctgagtttaataagaagagaaaagagttcttttgttatggctgaagtaatagagaaatgagctcgagtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttcctagggcgatcgccctcgaggcattacggcattacggcactcgcgagggtcccaattcgagcatggagccatttacaattgaatatatcctgccg

SEQ ID NO: 4 - DNA, 5858 bp, Artificial
  nucleotide sequence of T-DNA of pTJN48
aattacaacggtatatatcctgccagtactcggccgtcgaccgcggtaccccggaattaagcttgcatgcctgcaggattaccctgttatccctatggcgcgccgcatgcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcgctagcttagttcttcacgaacgtcgagagaacatcgaagtagtctgggaaagtctttctcgtgcaaccagggtctctgattgtaactggaacctcagcacatgcagcaagagagaatgccatagccatccgatgatcatcgtaggtatcgattgccgtaacgttgagcttttccggaggcgtaatgatgcagtaatccggtccttcctcaactgaagctccgagcttcgtaagctctgttctgatggcaaccattcgttcagtctccttgactctccacgaagcaacatctctgatagctgtcgggccatcggcaaacaaagcgacaacagcaagtgtcatggcaacgtctggcatcttgttcatgttcacgtcgatagccttgaggtgctttcttccaaatggctctctaggtggacctgtaacagtgactgaagtctctgtccaggtaacctttgcacccatcatctcaagaacctcagcgaacttgacatcaccttgaagactggtcgttccacaaccttcgactgtaactgttcctccagtgatagctgcaccagcaaggaagtaactagcagatgacgcatctccttcaacgtaagcgttcttcggactcttgtacttctgtcctcccttgatgtagaatctgtcccagctgtcagaatgttctgccttaactccgaatcgctccataagacgaagggtcatctcaacgtaggggatagagatgagcttgtcgatgatctcgatctcaacgtcacctaaagcaagtggtgcagccataagtagagcagacaagtactgactggagatggatccgctaagtttaacctttccacctggaagtcctccaattccgttgactctaacaggtgggcaatcagttccaaggaagcaatcaacatccgctcctagctgcttcaatccaacaacaaggtcaccgattggtctctctctcattctaggcacgccatcaagaacgtaagtggcatttcctccagcagcagtaacagcagcagtaagagatctcatggcgattcctgcgttaccgaggaaaagttgaacctcctccttggcatcttcaacagggaactttccaccacatcccacaacaacagctctctttgcagccttatccgcttcaacagaaagcccaagtgttctcaaggcacctagcatgtagtgaacgtcttcggagtttaggaggttgtcaacaactgtagtcccctcagatagagctgcaagaagcaagatgcggttggaaagagacttagacccaggtagcttaacggtaccgcttatctccttgatgggttggagaacgatctcttcagcacctgccatacatctgattctgcctccgtttgacacgttcccaagagatctggaactccttcttgcaaccggaagtgatgctgtagacttgagtccttggaatggagcaacagcagtagcagaagacgccatcataactgtcggagccatagacaaaggaggcaagtatgagagggtctcgaacttcttgttgccatatgctggccaaacttgcatacactgaactctacctccgttagagggtagggtactgaaatcgttagccttcttagttgtggggaaagctgcattggacttaagaccggtgaatggagcaaccatgttagcttgtgcaggtgcagttcttgaaaccgtcgcaacagatgagctgatggaagccatggttttggtttaataagaagagaaaagagttcttttgttatggctgaagtaatagagaaatgagctcgagtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttctagggcgatcgcttaattaaggatccggcgcgccgcatgccccgatcaaatctgagggacgttaaagcgatgataaattggaaccagaatatagaatctttgttctgctctagcttttcttctgtacattttttacgattagactatgattttcattcaataaccaaaattctgaagtttgtcatcaagttgctcaatcaaacttgtaccggtttgtttcggttttatatcagctcactgttacactttaaccaaaatcggtttatgtcttaataaaggaattgagtcggtttaactcatatccgtaccaatgcgacgtcgtgtccgcgtttcagtagctttgctcattgtcttctacgggaactttcccggacataggaaccgccctttcgttatcctcatccatcgtgaaatcaggaaataaatgttcgaagatttgaggtcaaaagtcgaatttcatgttgtctcttctatttagatacaaaattgaagcaattttcaccaatttaatgccaaaatttaaaacaacgctgataaagtgaaacttgattcgatttatatttcaaccgaaactgctgaagcaagaagaaaaagcgtaattacacataacaagaacgctaccgcaaactactaaacgccaaacccaatacaaaagtaaaacgcagacgcttaagtgagaaacccagaaaacacaaacgcggatcggggctagcttagttcttcacgaacgtcgagagaacatcgaagtagtctgggaaagtctttctcgtgcaaccagggtctctgattgtaactggaacctcagcacatgcagcaagagagaatgccatagccatccgatgatcatcgtaggtatcgattgccgtaacgttgagcttttccggaggcgtaatgatgcagtaatccggtccttcctcaactgaagctccgagcttcgtaagctctgttctgatggcaaccattcgttcagtctccttgactctccacgaagcaacatctctgatagctgtcgggccatcggcaaacaaagcgacaacagcaagtgtcatggcaacgtctggcatcttgttcatgttcacgtcgatagccttgaggtgctttcttccaaatggctctctaggtggacctgtaacagtgactgaagtctctgtccaggtaacctttgcacccatcatctcaagaacctcagcgaacttgacatcaccttgaagactggtcgttccacaaccttcgactgtaactgttcctccagtgatagctgcaccagcaaggaagtaactagcagatgacgcatctccttcaacgtaagcgttcttcggactcttgtacttctgtcctcccttgatgtagaatctgtcccagctgtcagaatgttctgccttaactccgaatcgctccataagacgaagggtcatctcaacgtaggggatagagatgagcttgtcgatgatctcgatctcaacgtcacctaaagcaagtggtgcagccataagtagagcagacaagtactgactggagatggatccgctaagtttaacctttccacctggaagtcctccaattccgttgactctaacaggtgggcaatcagttccaaggaagcaatcaacatccgctcctagctgcttcaatccaacaacaaggtcaccgattggtctctctctcattctaggcacgccatcaagaacgtaagtggcatttcctccagcagcagtaacagcagcagtaagagatctcatggcgattcctgcgttaccgaggaaaagttgaacctcctccttggcatcttcaacagggaactttccaccacatcccacaacaacagctctctttgcagccttatccgcttcaacagaaagcccaagtgttctcaaggcacctagcatgtagtgaacgtcttcggagtttaggaggttgtcaacaactgtagtcccctcagatagagctgcaagaagcaagatgcggttggaaagagacttagacccaggtagcttaacggtaccgcttatctccttgatgggttggagaacgatctcttcagcacctgccatacatctgattctgcctccgtttgacacgttcccaagagatctggaactccttcttgcaaccggaagtgatgctgtagacttgagtccttggaatggagcaacagcagtagcagaagacgccatcataactgtcggagccatagacaaaggaggcaagtatgagagggtctcgaacttcttgttgccatatgctggccaaacttgcatacactgaactctacctccgttagagggtagggtactgaaatcgttagccttcttagttgtggggaaagctgcattggacttaagaccggtgaatggagcaaccatgttagcttgtgcaggtgcagttcttgaaaccgtcgcaacagatgagctgatggaagccatggttttggatctgcgcatttaacaagaaattgaacagtcaattggggattttcattatccataactaaattttgaagaaattggaatactaaacgtcaccacttaaaaccctaatccagatgaatcgttatcgaaccagatataaccaaaaggggcaaaattgactcgaaaaccctagttctcgatacacggctaggtaatgacaatcgcacacagacaaatctggttatacagaacttcgaagcaagaaaaaaacgatgaagaatggatcatccaataaatcgactagactcaatcttcacaggtttatcgatccagcaaacttaaaagacggacctttattttcaaactggaatgggacaaaacccgaaactctattgtcgtaaaatcagatcgcggagacagtaacagaaaaaacattaaaaagtaatggaaagacctaaacccctgatctaattacaaacaaatcatacctgttcttcgcctgagtacgtatcgagagaaattgatgtctgtagaagaagaagaacggttaagagtagatttgggtgagaaagatgtgaaattgtttttataggcaaagacggagagtctattttttgagcaatcagatcgcatattaaatctaacggctgagatatcgatccgtgtgtacaataaaatgatgtataaaccgtcgatctgttttaatcgacggttcatattagtgatccgcgtgatggcagtgatagccactaagaatcgtcttttgttttacatgtggcgcctagggcgatcgccctcgaggcattacggcattacggcactcgcgagggtcccaattcgagcatggagccatttacaattgaatatatcctgccg

SEQ ID NO: 5 - DNA, 6340 bp, Artificial
  nucleotide sequence of T-DNA of pTJN49
aattacaacggtatatatcctgccagtactcggccgtcgaccgcggtaccccggaattaagcttgcatgcctgcaggattaccctgttatccctatggcgcgccgcatgcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcgctagcttagttcttcacgaacgtcgagagaacatcgaagtagtctgggaaagtctttctcgtgcaaccagggtctctgattgtaactggaacctcagcacatgcagcaagagagaatgccatagccatccgatgatcatcgtaggtatcgattgccgtaacgttgagcttttccggaggcgtaatgatgcagtaatccggtccttcctcaactgaagctccgagcttcgtaagctctgttctgatggcaaccattcgttcagtctccttgactctccacgaagcaacatctctgatagctgtcgggccatcggcaaacaaagcgacaacagcaagtgtcatggcaacgtctggcatcttgttcatgttcacgtcgatagccttgaggtgctttcttccaaatggctctctaggtggacctgtaacagtgactgaagtctctgtccaggtaacctttgcacccatcatctcaagaacctcagcgaacttgacatcaccttgaagactggtcgttccacaaccttcgactgtaactgttcctccagtgatagctgcaccagcaaggaagtaactagcagatgacgcatctccttcaacgtaagcgttcttcggactcttgtacttctgtcctcccttgatgtagaatctgtcccagctgtcagaatgttctgccttaactccgaatcgctccataagacgaagggtcatctcaacgtaggggatagagatgagcttgtcgatgatctcgatctcaacgtcacctaaagcaagtggtgcagccataagtagagcagacaagtactgactggagatggatccgctaagtttaacctttccacctggaagtcctccaattccgttgactctaacaggtgggcaatcagttccaaggaagcaatcaacatccgctcctagctgcttcaatccaacaacaaggtcaccgattggtctctctctcattctaggcacgccatcaagaacgtaagtggcatttcctccagcagcagtaacagcagcagtaagagatctcatggcgattcctgcgttaccgaggaaaagttgaacctcctccttggcatcttcaacagggaactttccaccacatcccacaacaacagctctctttgcagccttatccgcttcaacagaaagcccaagtgttctcaaggcacctagcatgtagtgaacgtcttcggagtttaggaggttgtcaacaactgtagtcccctcagatagagctgcaagaagcaagatgcggttggaaagagacttagacccaggtagcttaacggtaccgcttatctccttgatgggttggagaacgatctcttcagcacctgccatacatctgattctgcctccgtttgacacgttcccaagagatctggaactccttcttgcaaccggaagtgatgctgtagacttgagtccttggaatggagcaacagcagtagcagaagacgccatcataactgtcggagccatagacaaaggaggcaagtatgagagggtctcgaacttcttgttgccatatgctggccaaacttgcatacactgaactctacctccgttagagggtagggtactgaaatcgttagccttcttagttgtggggaaagctgcattggacttaagaccggtgaatggagcaaccatgttagcttgtgcaggtgcagttcttgaaaccgtcgcaacagatgagctgatggaagccatggttttggatctgcgcatttaacaagaaattgaacagtcaattggggattttcattatccataactaaattttgaagaaattggaatactaaacgtcaccacttaaaaccctaatccagatgaatcgttatcgaaccagatataaccaaaaggggcaaaattgactcgaaaaccctagttctcgatacacggctaggtaatgacaatcgcacacagacaaatctggttatacagaacttcgaagcaagaaaaaaacgatgaagaatggatcatccaataaatcgactagactcaatcttcacaggtttatcgatccagcaaacttaaaagacggacctttattttcaaactggaatgggacaaaacccgaaactctattgtcgtaaaatcagatcgcggagacagtaacagaaaaaacattaaaaagtaatggaaagacctaaacccctgatctaattacaaacaaatcatacctgttcttcgcctgagtttaataagaagagaaaagagttcttttgttatggctgaagtaatagagaaatgagctcgagtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttcctagggcgatcgcttaattaaggatccggcgcgccgcatgccccgatcaaatctgagggacgttaaagcgatgataaattggaaccagaatatagaatctttgttctgctctagcttttcttctgtacattttttacgattagactatgattttcattcaataaccaaaattctgaagtttgtcatcaagttgctcaatcaaacttgtaccggtttgtttcggttttatatcagctcactgttacactttaaccaaaatcggtttatgtcttaataaaggaattgagtcggtttaactcatatccgtaccaatgcgacgtcgtgtccgcgtttcagtagctttgctcattgtcttctacgggaactttcccggacataggaaccgccctttcgttatcctcatccatcgtgaaatcaggaaataaatgttcgaagatttgaggtcaaaagtcgaatttcatgttgtctcttctatttagatacaaaattgaagcaattttcaccaatttaatgccaaaatttaaaacaacgctgataaagtgaaacttgattcgatttatatttcaaccgaaactgctgaagcaagaagaaaaagcgtaattacacataacaagaacgctaccgcaaactactaaacgccaaacccaatacaaaagtaaaacgcagacgcttaagtgagaaacccagaaaacacaaacgcggatcggggctagcttagttcttcacgaacgtcgagagaacatcgaagtagtctgggaaagtctttctcgtgcaaccagggtctctgattgtaactggaacctcagcacatgcagcaagagagaatgccatagccatccgatgatcatcgtaggtatcgattgccgtaacgttgagcttttccggaggcgtaatgatgcagtaatccggtccttcctcaactgaagctccgagcttcgtaagctctgttctgatggcaaccattcgttcagtctccttgactctccacgaagcaacatctctgatagctgtcgggccatcggcaaacaaagcgacaacagcaagtgtcatggcaacgtctggcatcttgttcatgttcacgtcgatagccttgaggtgctttcttccaaatggctctctaggtggacctgtaacagtgactgaagtctctgtccaggtaacctttgcacccatcatctcaagaacctcagcgaacttgacatcaccttgaagactggtcgttccacaaccttcgactgtaactgttcctccagtgatagctgcaccagcaaggaagtaactagcagatgacgcatctccttcaacgtaagcgttcttcggactcttgtacttctgtcctcccttgatgtagaatctgtcccagctgtcagaatgttctgccttaactccgaatcgctccataagacgaagggtcatctcaacgtaggggatagagatgagcttgtcgatgatctcgatctcaacgtcacctaaagcaagtggtgcagccataagtagagcagacaagtactgactggagatggatccgctaagtttaacctttccacctggaagtcctccaattccgttgactctaacaggtgggcaatcagttccaaggaagcaatcaacatccgctcctagctgcttcaatccaacaacaaggtcaccgattggtctctctctcattctaggcacgccatcaagaacgtaagtggcatttcctccagcagcagtaacagcagcagtaagagatctcatggcgattcctgcgttaccgaggaaaagttgaacctcctccttggcatcttcaacagggaactttccaccacatcccacaacaacagctctctttgcagccttatccgcttcaacagaaagcccaagtgttctcaaggcacctagcatgtagtgaacgtcttcggagtttaggaggttgtcaacaactgtagtcccctcagatagagctgcaagaagcaagatgcggttggaaagagacttagacccaggtagcttaacggtaccgcttatctccttgatgggttggagaacgatctcttcagcacctgccatacatctgattctgcctccgtttgacacgttcccaagagatctggaactccttcttgcaaccggaagtgatgctgtagacttgagtccttggaatggagcaacagcagtagcagaagacgccatcataactgtcggagccatagacaaaggaggcaagtatgagagggtctcgaacttcttgttgccatatgctggccaaacttgcatacactgaactctacctccgttagagggtagggtactgaaatcgttagccttcttagttgtggggaaagctgcattggacttaagaccggtgaatggagcaaccatgttagcttgtgcaggtgcagttcttgaaaccgtcgcaacagatgagctgatggaagccatggttttggatctgcgcatttaacaagaaattgaacagtcaattggggattttcattatccataactaaattttgaagaaattggaatactaaacgtcaccacttaaaaccctaatccagatgaatcgttatcgaaccagatataaccaaaaggggcaaaattgactcgaaaaccctagttctcgatacacggctaggtaatgacaatcgcacacagacaaatctggttatacagaacttcgaagcaagaaaaaaacgatgaagaatggatcatccaataaatcgactagactcaatcttcacaggtttatcgatccagcaaacttaaaagacggacctttattttcaaactggaatgggacaaaacccgaaactctattgtcgtaaaatcagatcgcggagacagtaacagaaaaaacattaaaaagtaatggaaagacctaaacccctgatctaattacaaacaaatcatacctgttcttcgcctgagtacgtatcgagagaaattgatgtctgtagaagaagaagaacggttaagagtagatttgggtgagaaagatgtgaaattgtttttataggcaaagacggagagtctattttttgagcaatcagatcgcatattaaatctaacggctgagatatcgatccgtgtgtacaataaaatgatgtataaaccgtcgatctgttttaatcgacggttcatattagtgatccgcgtgatggcagtgatagccactaagaatcgtcttttgttttacatgtggcgcctagggcgatcgccctcgaggcattacggcattacggcactcgcgagggtcccaattcgagcatggagccatttacaattgaatatatcctgccg

SEQ ID NO: 6 - DNA, 7009 bp, Artificial
  nucleotide sequence of T-DNA of pTJN75
aattacaacggtatatatcctgccagtactcggccgtcgaccgcggtaccccggaattaagcttgcatgcctgcaggattaccctgttatccctatggcgcgccgcatgcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcgctagcttagttcttcacgaacgtcgagagaacatcgaagtagtctgggaaagtctttctcgtgcaaccagggtctctgattgtaactggaacctcagcacatgcagcaagagagaatgccatagccatccgatgatcatcgtaggtatcgattgccgtaacgttgagcttttccggaggcgtaatgatgcagtaatccggtccttcctcaactgaagctccgagcttcgtaagctctgttctgatggcaaccattcgttcagtctccttgactctccacgaagcaacatctctgatagctgtcgggccatcggcaaacaaagcgacaacagcaagtgtcatggcaacgtctggcatcttgttcatgttcacgtcgatagccttgaggtgctttcttccaaatggctctctaggtggacctgtaacagtgactgaagtctctgtccaggtaacctttgcacccatcatctcaagaacctcagcgaacttgacatcaccttgaagactggtcgttccacaaccttcgactgtaactgttcctccagtgatagctgcaccagcaaggaagtaactagcagatgacgcatctccttcaacgtaagcgttcttcggactcttgtacttctgtcctcccttgatgtagaatctgtcccagctgtcagaatgttctgccttaactccgaatcgctccataagacgaagggtcatctcaacgtaggggatagagatgagcttgtcgatgatctcgatctcaacgtcacctaaagcaagtggtgcagccataagtagagcagacaagtactgactggagatggatccgctaagtttaacctttccacctggaagtcctccaattccgttgactctaacaggtgggcaatcagttccaaggaagcaatcaacatccgctcctagctgcttcaatccaacaacaaggtcaccgattggtctctctctcattctaggcacgccatcaagaacgtaagtggcatttcctccagcagcagtaacagcagcagtaagagatctcatggcgattcctgcgttaccgaggaaaagttgaacctcctccttggcatcttcaacagggaactttccaccacatcccacaacaacagctctctttgcagccttatccgcttcaacagaaagcccaagtgttctcaaggcacctagcatgtagtgaacgtcttcggagtttaggaggttgtcaacaactgtagtcccctcagatagagctgcaagaagcaagatgcggttggaaagagacttagacccaggtagcttaacggtaccgcttatctccttgatgggttggagaacgatctcttcagcacctgccatacatctgattctgcctccgtttgacacgttcccaagagatctggaactccttcttgcaaccggaagtgatgctgtagacttgagtccttggaatggagcaacagcagtagcagaagacgccatcataactgtcggagccatagacaaaggaggcaagtatgagagggtctcgaacttcttgttgccatatgctggccaaacttgcatacactgaactctacctccgttagagggtagggtactgaaatcgttagccttcttagttgtggggaaagctgcattggacttaagaccggtgaatggagcaaccatgttagcttgtgcaggtgcagttcttgaaaccgtcgcaacagatgagctgatggaagccatggttttggatctgcgcatttaacaagaaattgaacagtcaattggggattttcattatccataactaaattttgaagaaattggaatactaaacgtcaccacttaaaaccctaatccagatgaatcgttatcgaaccagatataaccaaaaggggcaaaattgactcgaaaaccctagttctcgatacacggctaggtaatgacaatcgcacacagacaaatctggttatacagaacttcgaagcaagaaaaaaacgatgaagaatggatcatccaataaatcgactagactcaatcttcacaggtttatcgatccagcaaacttaaaagacggacctttattttcaaactggaatgggacaaaacccgaaactctattgtcgtaaaatcagatcgcggagacagtaacagaaaaaacattaaaaagtaatggaaagacctaaacccctgatctaattacaaacaaatcatacctgttcttcgcctgagtttaataagaagagaaaagagttcttttgttatggctgaagtaatagagaaatgagctcgagtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttcctagggcgatcgcttaattaaggatccggcgcgccgcatgccccgatcaaatctgagggacgttaaagcgatgataaattggaaccagaatatagaatctttgttctgctctagcttttcttctgtacattttttacgattagactatgattttcattcaataaccaaaattctgaagtttgtcatcaagttgctcaatcaaacttgtaccggtttgtttcggttttatatcagctcactgttacactttaaccaaaatcggtttatgtcttaataaaggaattgagtcggtttaactcatatccgtaccaatgcgacgtcgtgtccgcgtttcagtagctttgctcattgtcttctacgggaactttcccggacataggaaccgccctttcgttatcctcatccatcgtgaaatcaggaaataaatgttcgaagatttgaggtcaaaagtcgaatttcatgttgtctcttctatttagatacaaaattgaagcaattttcaccaatttaatgccaaaatttaaaacaacgctgataaagtgaaacttgattcgatttatatttcaaccgaaactgctgaagcaagaagaaaaagcgtaattacacataacaagaacgctaccgcaaactactaaacgccaaacccaatacaaaagtaaaacgcagacgcttaagtgagaaacccagaaaacacaaacgcggatcggggctagcttagttcttcacgaacgtcgagagaacatcgaagtagtctgggaaagtctttctcgtgcaaccagggtctctgattgtaactggaacctcagcacatgcagcaagagagaatgccatagccatccgatgatcatcgtaggtatcgattgccgtaacgttgagcttttccggaggcgtaatgatgcagtaatccggtccttcctcaactgaagctccgagcttcgtaagctctgttctgatggcaaccattcgttcagtctccttgactctccacgaagcaacatctctgatagctgtcgggccatcggcaaacaaagcgacaacagcaagtgtcatggcaacgtctggcatcttgttcatgttcacgtcgatagccttgaggtgctttcttccaaatggctctctaggtggacctgtaacagtgactgaagtctctgtccaggtaacctttgcacccatcatctcaagaacctcagcgaacttgacatcaccttgaagactggtcgttccacaaccttcgactgtaactgttcctccagtgatagctgcaccagcaaggaagtaactagcagatgacgcatctccttcaacgtaagcgttcttcggactcttgtacttctgtcctcccttgatgtagaatctgtcccagctgtcagaatgttctgccttaactccgaatcgctccataagacgaagggtcatctcaacgtaggggatagagatgagcttgtcgatgatctcgatctcaacgtcacctaaagcaagtggtgcagccataagtagagcagacaagtactgactggagatggatccgctaagtttaacctttccacctggaagtcctccaattccgttgactctaacaggtgggcaatcagttccaaggaagcaatcaacatccgctcctagctgcttcaatccaacaacaaggtcaccgattggtctctctctcattctaggcacgccatcaagaacgtaagtggcatttcctccagcagcagtaacagcagcagtaagagatctcatggcgattcctgcgttaccgaggaaaagttgaacctcctccttggcatcttcaacagggaactttccaccacatcccacaacaacagctctctttgcagccttatccgcttcaacagaaagcccaagtgttctcaaggcacctagcatgtagtgaacgtcttcggagtttaggaggttgtcaacaactgtagtcccctcagatagagctgcaagaagcaagatgcggttggaaagagacttagacccaggtagcttaacggtaccgcttatctccttgatgggttggagaacgatctcttcagcacctgccatacatctgattctgcctccgtttgacacgttcccaagagatctggaactccttcttgcaaccggaagtgatgctgtagacttgagtccttggaatggagcaacagcagtagcagaagacgccatcataactgtcggagccatagacaaaggaggcaagtatgagagggtctcgaacttcttgttgccatatgctggccaaacttgcatacactgaactctacctccgttagagggtagggtactgaaatcgttagccttcttagttgtggggaaagctgcattggacttaagaccggtgaatggagcaaccatgttagcttgtgcaggtgcagttcttgaaaccgtcgcaacagatgagctgatggaagccatggttttggatctgcgcatttaacaagaaattgaacagtcaattggggattttcattatccataactaaattttgaagaaattggaatactaaacgtcaccacttaaaaccctaatccagatgaatcgttatcgaaccagatataaccaaaaggggcaaaattgactcgaaaaccctagttctcgatacacggctaggtaatgacaatcgcacacagacaaatctggttatacagaacttcgaagcaagaaaaaaacgatgaagaatggatcatccaataaatcgactagactcaatcttcacaggtttatcgatccagcaaacttaaaagacggacctttattttcaaactggaatgggacaaaacccgaaactctattgtcgtaaaatcagatcgcggagacagtaacagaaaaaacattaaaaagtaatggaaagacctaaacccctgatctaattacaaacaaatcatacctgttcttcgcctgagtacgtatcgagagaaattgatgtctgtagaagaagaagaacggttaagagtagatttgggtgagaaagatgtgaaattgtttttataggcaaagacggagagtctattttttgagcaatcagatcgcatattaaatctaacggctgagatatcgatccgtgtgtacaataaaatgatgtataaaccgtcgatctgttttaatcgacggttcatattagtgatccgcgtgatggcagtgatagccactaagaatcgtcttttgttttacatgtggcgccacaaattagggtaatgaagcggcaatattttggaactcggaaaataaaattgcgccatcacattatttgaaaattttcacatgcttttattttaaaaacccacgaattacaagttacaaccgaaaaagatttataatatagtgatttatactaattttgtagtagcttaatgtatattgatactggaaaaacaatgacaatcatatgttagtattatcaagttatcgtattgatattgatattggaacatacaatgggtattgccttctttcgaccataaatatcaccaaatttacaaagtttgtgtataccaagttatcaattgtaaatgggatgtcaacattttaatttccctttgagaaactatagaccacaagaacacacttcaatagataaagtaactatttacataagaggttttaaaatcacattaacaaaaataattaccaaccggcactcacaaatacaaacagagcacacgacatgtcaaagccacaagtaaattcgttgagtggtggtttcattacaattgtgtcacttgcagcacaaactatcttgctctgggaatcatctcagcatcaaagatcatgctcacttcaggggaacttagtgtatccatgcctcgactcatatttctcctcgacctgcaggcatgcaagcttttaaaaccaattgttcgaacgtacgtcgcgactcgacctgcaggaattctagatacgtagcgatcgccatggagccatttacaattgaatatatcctgccg

SEQ ID NO: 7 - protein, 83 aa, Artificial
  aminoacid sequence of the optimized transit peptide, containing sequences of the RuBisCO small subunit of Zea mays (corn) and Helianthus annuus (sunflower), as described by Lebrun et al. (1999)
MRRSKTMASISSSVATVSRTAPAQANMVAPFTGLKSNAAFPTTKKANDFSTLPSNGGRVQCMQVWPAYGNKKFETLSYLPPLS

SEQ ID NO: 8 - protein, 492 aa, Artificial
  aminoacid sequence of the double-mutant 5-enol-pyruvylshikimate-3-phosphate synthase of Zea mays (corn) (Lebrun et al., 1997)
MAPTVMMASSATAVAPFQGLKSTASLPVARRSSRSLGNVSNGGRIRCMAGAEEIVLQPIKEISGTVKLPGSKSLSNRILLLAALSEGTTVVDNLLNSEDVHYMLGALRTLGLSVEADKAAKRAVVVGCGGKFPVEDAKEEVQLFLGNAGIAMRSLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISIPYVEMTLRLMERFGVKAEHSDSWDRFYIKGGQKYKSPKNAYVEGDASSASYFLAGAAITGGTVTVEGCGTTSLQGDVKFAEVLEMMGAKVTWTETSVTVTGPPREPFGRKHLKAIDVNMNKMPDVAMTLAVVALFADGPTAIRDVASWRVKETERMVAIRTELTKLGASVEEGPDYCIITPPEKLNVTAIDTYDDHRMAMAFSLAACAEVPVTIRDPGCTRKTFPDYFDVLSTFVKN

SEQ ID NO: 9 - DNA, 4833 bp, Artificial
  nucleotide sequence of the Arabidopsis thaliana H3 gene 1 and H3 gene 2 for H3.3-like histone variant
gatctgcattaacttccgtccgccaatttctgccctttgccaatccatatctgaagaagaaaaaaaaacacaaaattacgatttagatccgatataacaaaatttgaatcgcacagatcgatctctttggagattctatacctagaaaatggagacgattttcaaatctctgtaaaaattctggtttcttcttgacggaagaagacgacgactccaatatttcggttagtactgaaccggaaagtttgactggtgcaaccaatttaatgtaccgtacgtaacgcaccaatcggattttgtattcaatgggccttatctgtgagcccattaattgatgtgacggcctaaactaaatccgaacggtttatttcagcgatccgcgacggtttgtattcagccaatagcaatcaattatgtagcagtggtgatcctcgtcaaaccagtaaagctagatctggaccgttgaattggtgcaagaaagcacatgttgtgatatttttacccgtacgattagaaaacttgagaaacacattgataatcgataaaaaccgtccgatcatataaatccgctttaccatcgttgcctataaattaatatcaatagccgtacacgcgtgaagactgacaatattatctttttcgaattcggagctcaagtttgaaattcggagaagctagagagttttctgaggtacgattcttcgatcctctttgattttcctggaaatattttttcggtgatcgtgaaactactggaatcgctcgataggtggtacgaaattaggcgagattagtttctattcttggccattatcttgtttcttcgccgaatgatcttccgtataaagattttaggttagagatgaatcgtatagctagatttcatcaccagatagtttctttgtctagaatctctgaaattctcgatagttttcacatgtgtaaatagattgttcttattcggcgattgttgattagggttttgattttcttgattatgcgattgcaattagggattttctttggttttgtgttgatcttacgatacattcctgcaattgaatacgtatggatctaaatcttgttaatttgttgaacagatggctcgtacaaagcaaacagctcgtaagtctactggaggaaaggctcctaggaagcagcttgctacaaaggtaagactcgggctctcacatgtgatctgagtagcttgataaacacatttctagatttgttctaattggtggatgttttaatttaaggctgcacgtaagtctgcaccaaccactggaggagtcaagaagccccatcgttaccgtccaggaactgttgcactacggtatgcaatctgtttcttccgcaaattcaattgtgtttttactttattgatatcgtcattgattaaactctgttttattgatcttgattgacagtgaaattcgtaagtaccagaagagtaccgagttgctgatcaggaagctccctttccagaggctagttcgtgagattgcccaggatttcaaggtaatgtgtctgttcgtctgattcagtctacaaatggatttaactttttacttgttttgtcataaccttgtgttcttatcttgtctctttgattgcagactgacttgcgtttccagagccatgctgtgcttgcactccaggaggctgctgaagcataccttgtgggtctctttgaggacactaacctctgcgccatccatgccaagcgtgtgaccataatgcccaaagacattcagctcgctcgcaggatcagaggagaacgtgcttaattgtgattgggaagtagttggattatgtgaatgtgggtggttttctaaatagatagttggaattagataatggcttaaaaagcttttgggtgtaggagacttggaaacaatggtgttttttttttcttttctttctgtttcttgtctagtcggtttatgatttgcttgtgttcttcttagctacgttttgtggtgtgtattgctttcttctttggcaatgttgaacaccttttgttgttgaattcatctctgaaacatatgaatatgttaaaagtttctgactcttgtttcatctgtatctgttcgatattgtatgattgagttttattgtttattaataggctttctgtttcaaagttcttttcaatatcaacaacttttacgttcacgaaattggtgatccgtgtgacacttaaaatctctaatcgtggccgttgggctgtgttgagaaaacacaatgtacaatagtttgaaccgtagataaagaatgtgagaaagcacaaaggagcgtatcgtgataaccctttcgtgagtattataaataacaattcaactacattgcggccacaaccactacttcaatcatcccctattactgcttttgttacgaaagacaaaaaaacaaaacaaaacaaaagtcagctcaggcgaagaacaggtatgatttgtttgtaattagatcaggggtttaggtctttccattactttttaatgttttttctgttactgtctccgcgatctgattttacgacaatagagtttcgggttttgtcccattccagtttgaaaataaaggtccgtcttttaagtttgctggatcgataaacctgtgaagattgagtctagtcgatttattggatgatccattcttcatcgtttttttcttgcttcgaagttctgtataaccagatttgtctgtgtgcgattgtcattacctagccgtgtatcgagaactagggttttcgagtcaattttgccccttttggttatatctggttcgataacgattcatctggattagggttttaagtggtgacgtttagtattccaatttcttcaaaatttagttatggataatgaaaatccccaattgactgttcaatttcttgttaaatgcgcagatggctcgtaccaagcaaaccgctcgtaagtccaccggaggtaaagctccaaggaagcaacttgctactaaggttttgttccttcttgtctctttttcaaataatacttgtgttgtgaagttgaatgttaatctccttctttattaacctcaggctgctcgtaaatctgcaccaactactggtggagtcaagaaaccacatcgttaccgtcctggaactgttgctctccggtttgtcccttcttcgatttgtatgtgattctttgagattatgtaacattgtgtgttaacattcctcttatcttttggtgttcagtgaaatccgtaagtaccagaagagtactgagttgcttatcaggaaactgccatttcagaggctagtccgtgagattgctcaagatttcaagactgatttgcgtttccagagccatgctgtcttagctctccaggaagctgcagaagcatatcttgttggtctctttgaagacactaacctttgtgccattcatgccaagcgtgtgaccataatgcccaaagacattcagctcgctcgtcgtatcagaggtgaacgcgcttaagccaaccaagaatccgagatttggttcaagtagtttttgttttttatgaaagcaagatcttaattgctgtgttttttaaatctctgggtatgtagtagtaagagtagtaagaagtctgcaatctaagaatgttgtgttctttaagcttattattatgtgtgttgcgaactctttttaactcttttgttaaattcttatgttttcttaactcgtttgttcgtcaccattgctttctcttcaaataatggctgatttttttctataattttggatttgttgaatgctttcttttaaaagaatcagattttggacttttgaccaaagaaaataataatatcagacgataaaatagacggctctcgataaaactaaccctaaaaataaggaaataagttcctctttgaaccaaattttctttctttgaccaatagatctttttgtcaacctcttaaatatattcttagtcaatcttctaataaacccattggccattaccaaaaattcctcagaaacgctgaataaaaaacattctatcatctatgggagaagtcgatgagaaaccgtccattgatgtagcttctccaaggtattcatcgaacaaagttgcggatattggtacagagctttacaaaatgaaagcttcccttgagaacagagaaaacgaagtcgtttctttaaaacaagagttattgaagaaagacatcttcatcaagaatcttgaagctgcagagaagaaactgcttgattcgtttaaagatcaatcgagagagttagaggaaaccaaagctttggtagaagagtcaaaggtagagattgcttcattaaaagagaagatagatacatcttacaatagccaagattccagcgaggaagacgaagacgatagttctgttcaggattttgatattgagtctttgaagactgagatggaatcgaccaaggagagtcttgcacaggctcatgaggctgcacaagcctcttctttaaaggtttctgaattgttagaagagatgaaatcggttaagaacgagcttaaatcagcgactgatgcagagatgactaacgagaaagcaatggatgatttggctttagctttgaaagaggtagctactgattgtagccaaacgaaagagaagcttgtgattgtggaaacagagctcgaggctgctaggttagagtctcagcaatggaaggacaagtacgaggaagtgaggaaagatgctgaattacttaagaacacgagtgagagactaaggattgaagcagaggaatcgcttttggcttggaatgggaaagaatctgtgtttgtgacttgtataaagagaggagaagatgagaagaattc

SEQ ID NO: 10 - DNA, 1676 bp, Artificial
  nucleotide sequence of the Medicago sativa cultivar Chief histone H3.2 gene
acttnactaacggagtctgcatttaggtactaaaatgactaatatagtctacattcagggactattttgcaatttacctgcattcagggactaaagtgacgacttctttcctattcagagactaaagtgaccaatctctcaaaatgaagatattttgttttgttttggcgagataagttgcacacgatttacactcacaaaagaaacacaaattgtccacgctggcaatccgcaactttacaaaccaaccaatcagaaacaaacacacggatcgcacttaatattttcacttaaaaaactcatcattaccgttgaagcattcaaagtccacgatctctctactctaattaaccttccttaatcatcattaacccttgcatatataaacacacttctcttcaacaaccctcattacacatttcttctctttcgctaaatctaatcaatctttccctctcttcgagctttctctctccgattccatggctcgtaccaagcaaaccgctcgcaaatccactggtggtaaggctccaaggaagcagctcgccaccaaggtaaccaccgttcaccgccgtaacggttttttcttctttctgttttcttgatcttagggtttcgttttcttcaattcgaattttttgattgatttcatcgattttttggttcaggctgctaggaaatctgctcctactactggaggagtcaagaaacctcaccgataccgccctggaactgtcgctcttcggtaatttccttttccccaatttttaggttttcggagttttgcagtttctattattaattttttttaggttttcgttgtgttttgaatattctattgaattttatgttnngaatttgaattttggattcataatttttaggaatttggagttttgtattctcggtttatgatttttaggttttcgtagttgttaattttcaattgttgtgatttacagtgagatccgtaagtaccagaagagtaccgagcttttgatccgcaagcttccatttcagcgtcttgtccgtgaaattgctcaagatttcaaggtaaatattgttacttagtttcataattgattttgtgtgaaatcttgttcctctttggtttatttaatttgtaattgttgtttttgatatttttctgattaatttgctgttgttgttatagacggatctgagattccagagccatgcagttcttgcacttcaggaagcagctgaggcttacctggttggattgtttgaggacaccaatctgtgtgcaattcatgctaagagggtgacaattatgcctaaggacattcagcttgctcgtcgcattcgcggtgaacgtgcttagggtggtgaaggcgcttttagcgttatggtggattagtattttggaaggatttagggttttatgaattgaattttcttttatgcgttgtatagttctgaacctataatgttcaatctttaacaacagacatattttggattatgattagttttttgcggacaaatttgtgatgtaattggtcaattacaattgaagtctctgcaactattttacttatatctccattgcttcctgatttcgttatgcgcttttgatgatgcgactgtggtttctagctctgaattcaatttgtgatgcgtttctctctatggtagtttgtctatcgctgaca